(19) United States
(12) Patent Application Publication
(10) Pub. No.: US 2021/0171910 A1
(43) Pub. Date: Jun. 10, 2021

(54) CHIMERIC ANTIGEN RECEPTOR FIBROBLAST CELLS FOR TREATMENT OF CANCER

(71) Applicant: Figene, LLC, Houston, TX (US)

(72) Inventors: Pete O'Heeron, Houston, TX (US); Thomas Ichim, San Diego, CA (US)

(21) Appl. No.: 17/271,529

(22) PCT Filed: Aug. 27, 2019
(86) PCT No.: PCT/US2019/048311

§ 371 Date: Feb. 25, 2021

Related U.S. Application Data
(60) Provisional application No. 62/820,636, filed on Mar. 19, 2019.
(60) Provisional application No. 62/723,105, filed on Aug. 27, 2018.

(51) Int. Cl. C12N 5/077; C07K 14/705; C07K 14/54; A61K 45/06; A61P 35/00

(52) U.S. Cl. CPC C12N 5/0656; C07K 14/705; C07K 14/5418; A61P 35/00; C07K 14/5434; A61K 45/06; C07K 14/5443

(57) ABSTRACT

Embodiments of the disclosure include methods and compositions related to fibroblasts that express at least one chimeric antigen receptor (CAR). In specific embodiments, the chimeric antigen receptor has an antigen-specific targeting region that targets a tumor or pathogen antigen and that comprises an intracellular domain that upon signal through the chimeric antigen receptor is able to endow the fibroblasts with the ability to induce an immune response in an individual. In other embodiments, activity of CAR-expressing cells of any kind is enhanced through administration of fibroblasts that may or may not themselves express one or more CARs.

The present application claims priority to U.S. Provisional Application Ser. No. 62/723,105, filed Aug. 27, 2018, and to U.S. Provisional Application Ser. No. 62/820,636, filed Mar. 19, 2019, both of which applications are incorporated by reference herein in their entirety.

### TECHNICAL FIELD

Embodiments of the disclosure include at least the fields of cell therapy, molecular biology, cell biology, and medicine.

### BACKGROUND

In the area of cancer immunotherapy, the use of CAR-T cells has led to a revolution in treatment. The original concept of antitumor immunity began in part by observations of tumor infiltrating lymphocytes, that is, in a wide variety of tumors, lymphocytic infiltration was observed and associated with positive prognosis. This has been observed in bowel tumors [1], head and neck cancer [2, 3], bladder cancer [4-6], glioblastoma [7, 8], breast cancer [5], melanoma [9, 10], lung cancer [11, 12], stomach cancer [13], ovarian cancer [14-16], and colorectal cancer [17, 18].

In some situations it has been demonstrated that immune stimulation through vaccination or immunotherapy results in augmentation of tumor infiltrating lymphocytes [19-22]. Furthermore, in patients who undergo spontaneous regressions of cancer, the regressions are associated with lymphocytic infiltrates [23-40]. Furthermore, in some patients spontaneous regression occurs after bacterial or viral infections [41], further suggesting immunological causes. In addition to lymphocytic infiltrations, antigen-specific T cells have been detected to be associated with spontaneous regression [42]. Studies initiated by Rosenberg's group demonstrated that extraction of tumor infiltrating lymphocytes followed by ex vivo expansion and re-infusion results in substantial tumor regression [43-48], especially when patients are previously treated by lymphodepletion.

Augmentation of activity of lymphocyte immunotherapies was observed utilizing chimeric receptors in animal studies, Hwu et al examined the in vivo activity of murine T cells transduced with a chimeric receptor gene (MOv-gamma) derived from the mAb MOv18, which binds to a folate-binding protein overexpressed on most human ovarian adenocarcinomas. Nude mice that were given i.p. implants of human ovarian cancer (IGROV) cells were treated 3 days later with i.p. murine tumor-infiltrating lymphocytes (TIL) derived from an unrelated tumor. Mice treated with MOv-gamma-transduced TIL (MOv-TIL) had significantly increased survival compared to mice treated with saline only, nontransduced TIL, or TIL transduced with a control anti-trinitrophenyl chimeric receptor gene (TNP-TIL). In another model, C57BL/6 mice were given i.v. injections of a syngeneic methylcholanthrene-induced sarcoma transduced with the folate-binding protein (FBP) gene [49]. Three days later, mice were treated i.v. with various transduced murine TIL (derived from an unrelated tumor), followed by low-dose systemic interleukin 2. Eleven days after tumor injection, mice were sacrificed, and lung metastases were counted. In multiple experiments, mice receiving MOv-TIL had significantly fewer lung metastases than did mice treated with interleukin 2 alone, nontransduced TIL, or TNP-TIL. These studies indicate that T cells can be gene modified to react in vivo against tumor antigens, defined by mAbs. This approach is potentially applicable to a number of neoplastic and infectious diseases and may allow adoptive immunotherapy against types of cancer not previously amenable to cellular immunotherapy [50].

Researchers have attempted to counter the immune system's tolerance to cancer cell antigens by genetically modifying T cells with a chimeric antigen receptor (CAR) via grafting, called CAR-T cells [51]. CAR-T cells have the advantage of not requiring presentation of tumor antigen on MHC because they possess an antibody domain. CAR are usually generated by joining a single chain antibody (scFv) to an intracellular signaling domain, usually the zeta chain of the TCR/CD3 complex. The most recent construction of CARs also contain a co-stimulatory molecule such as CD28 or 4-1BB that can improve effector cell survival and proliferation [52]. For cancer therapy, CARs have at least three major advantages over natural T cell receptors. First, the antigen binding affinity of scFv is typically much higher than the binding moiety of most TCRs. A high affinity binding is desired for efficient T cell activation. Second, because of the nature of scFv-mediated antigen binding, CAR recognition is non-MHC restricted and independent of antigen processing. This widens the use of CARs to patients with different MHC haplotypes. Third, because CAR recognition is non-MHC restricted, their ability to target cancer cells is not hampered by a cancer cell's ability to down regulate MHC (an important mechanism by which tumor cells evade cancer immunotherapies). CARs have been previously constructed with scFvs that bind to a variety of tumor-associated antigens. Encouraging preclinical data has prompted a series of clinical trials using adoptive transfer of T cells engrafted with these CARs for treatment of tumors having different tissue origins, including melanoma, lymphoma, neuroblastoma, and colorectal cancer. Many of these trials have shown promising results, even complete remission of the established tumors in some cases.

Despite the impressive improvement of CAR-T cells over native T effector cells, there are significant drawbacks. For example, CAR-T cells do not actively migrate to the tumor site and they lack an active mechanism to extravasate into tumor tissue. Unfortunately, current CAR-T cell approaches are limited by the lack of ability to generate “universal donor” CAR-T cells, as well as by general toxicity in some cases or lack of efficacy in others.

The present disclosure provides solutions to deficiencies in the art of cell therapy.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### Brief Summary

The present disclosure is directed to methods and compositions that concern cell therapy for an individual in need thereof. In particular cases, the individual is a mammal, including a human, dog, cat, horse, and so forth. The individual may have a disease or medical condition for which the cell therapy is effective, including for amelioration of at least one symptom. The disclosure also includes methods of preventing any disease or medical condition, such as for an individual having an elevated risk for the disease or medical condition compared to another (for example, having a personal or family history, having a genetic marker associated with the disease or medical condition, being a smoker and/or obese, and so forth). The methods and compositions relate to any disease or medical condition having at least one associated cell antigen to which an antibody of any kind may target. The therapy may remove the symptom, reduce the severity of the symptom, and/or delay the onset of the symptom. In specific embodiments, the individual has cancer or is at risk for having cancer.

In particular embodiments, the disclosure concerns fibroblasts that express one or more chimeric antigen receptors (CARs) having at least one extracellular domain that comprises at least one antigen-specific targeting region, a transmembrane domain, and at least one endodomain that comprises one or more intracellular signaling domains. The extracellular domain may comprise an antigen-specific targeting region that comprises an antibody or functional fragment thereof. In specific embodiments, the CAR lacks costimulatory domains, such as CD28, 4-1BB, OX40, and others.

The present disclosure in certain embodiments pertains to antitumor immunity, including induction of antitumor immunity utilizing CAR-transfected fibroblasts to become activated upon or subsequent to contact with a cancer cell, including a cancer cell in a tumor. In specific embodiments, the activation induces Type 1 immunity and/or tumor inhibition.

In particular embodiments, the cell therapy comprises modified fibroblast cells and their uses. The fibroblasts in particular are modified by the hand of man to express one or more chimeric antigen receptors. Any chimeric antigen receptor in the fibroblasts may be mono-specific for an antigen, bi-specific for two non-identical antigens, tri-specific for three non-identical antigens, and so forth. In specific cases, the chimeric antigen receptor targets an antigen, such as a tumor antigen or viral antigen or bacterial antigen or fungal antigen or parasitic antigen, for example.

Embodiments of the disclosure encompass cells that express one or more chimeric antigen receptors and methods of using the cells that express the one or more chimeric antigen receptors. Methods include delivery to an individual in need thereof of a therapeutically effective amount of cells that express one or more chimeric antigen receptors. Also encompassed in the disclosure are kits that comprise the modified cells and/or reagents to generate them. In some embodiments, the cells that express one or more chimeric antigen receptors are fibroblasts. In some embodiments, the cells that express one or more chimeric antigen receptors are immune cells, including cytotoxic immune cells such as T cells, NK cells, and NKT cells. In some embodiments, immune cells expressing at least one chimeric antigen receptor are administered to an individual before, with, after, or a combination thereof administration of fibroblasts, including any fibroblast encompassed herein.

Embodiments of the disclosure include fibroblast cells that express a chimeric antigen receptor (CAR). In specific embodiments, the chimeric antigen receptor comprises at least one antigen-specific targeting region, at least one transmembrane domain, and at least one intracellular signaling domain. The antigen-specific targeting region may be an scFv. Any antigen-specific targeting region may target CD19, TEM-1, TEM-2, TEM-3, TEM-4, TEM-5, TEM-6, TEM-7, TEM-8, ROBO-4, VEGRF2, CD109, survivin and/or CD93 antigen, for example. The intracellular signaling domain may comprise part or all of an intracellular domain of TLR-4. The intracellular signaling domain may comprise a shRNA that encodes a transcript that generates at least one siRNA capable of inhibiting expression of HLA I and/or HLA II.

Any fibroblast cell encompassed herein may comprise activity to elicit an immune reaction in an individual. In some cases, the activity comprises the ability to produce cytokine production, for example over the level of fibroblasts lacking expression of the CAR. Particular cytokines include one or more of IL-12, IL-7, IL-15 and IL-21, or a combination thereof. The activity may comprise the ability to stimulate a T cell response, NKT cell response, and/or an NK cell response, and the NK cells may be CD94⁺ and/or CD117⁺ and/or CD161⁻ and/or NKG2D⁺ and/or NKp46⁺ and/or CD226⁺ and/or CD57⁺. The cell(s) may have been exposed to one or more anti-apoptotic proteins, such as STC-1, BCL-2, XIAP, Survivin, Bcl-2XL, GATA-4, FGF-2, HO-1, or a combination thereof. The cells may further expresses one or more anti-apoptotic proteins, such as STC-1, BCL-2, XIAP, Survivin, Bcl-2XL, GATA-4, FGF-2, HO-1, or a combination thereof.

The fibroblast cells, including CAR-expressing fibroblasts and fibroblasts not expressing a CAR, may be from the skin, heart, blood vessels, bone marrow, skeletal muscle, liver, pancreas, brain, adipose tissue, foreskin, placental, and/or umbilical cord. The cells may be placental, fetal, neonatal, adult, or a mixture thereof. The fibroblast cells may be mammalian, including human. The fibroblast cells may be autologous, allogenic, xenogenic, or a combination thereof to an individual, such as the individual administered the fibroblast cells.

Any cells may be comprised in an isolated plurality.

Embodiments of the disclosure include methods of treating a medical disease or condition in an individual, comprising the step of providing or administering to the individual a therapeutically effective amount of a plurality of the fibroblast cells encompassed by the disclosure. The medical disease or condition may be cancer, in some cases. In specific embodiments, an individual with cancer is provided an effective amount of fibroblast cells and may be given an additional cancer therapy, which may be surgery, radiation, chemotherapy, hormone therapy, immune therapy, or a combination thereof. In some embodiments, the individual is provided CAR-expressing cells, such as CAR-expressing T cells, NK cell, NKT cells, fibroblasts, or a combination thereof, before, during, after, or a combination thereof with the administration of fibroblasts, including modified and/or unmodified fibroblasts.

In some embodiments, an individual is provided an effective amount of one or more immune stimulators, such as a toll like receptor agonist.

Embodiments of the disclosure concern methods for stimulating T cells, NKT cells and/or NK cells. The methods for stimulating T cells, NKT cells and/or NK cells may comprise providing the T cells, NKT cells and/or NK cells with an effective amount of any of the CAR-expressing fibroblasts encompassed herein. The stimulation may occur ex vivo or may occur in vivo in an individual, including any individual encompassed herein. In some embodiments, CAR-expressing cells, including CAR-expressing immune cells, CAR-expressing stem cells, CAR-expressing fibroblasts, or a combination thereof, in an individual may be enhanced by providing the individual an effective amount of fibroblasts. The fibroblasts may be administered to the individual before, during, and/or after administering the CAR-expressing cells to the individual.

In some embodiments, the CAR-expressing cells encompassed herein, including CAR-expressing immune cells, CAR-expressing stem cells, CAR-expressing fibroblasts, or a combination thereof, may be modified to express one or more cytokines. Any methods of the disclosure may comprise the step of preparing the cells.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWING

The following drawing forms part of the present specification and are included to further demonstrate certain aspects of the present disclosure. The subject matter of the disclosure may be better understood by reference to the drawing in combination with the detailed description of specific embodiments presented herein.

FIG. 1 shows suppression of leukemia cell proliferation by CAR-expressing fibroblasts.

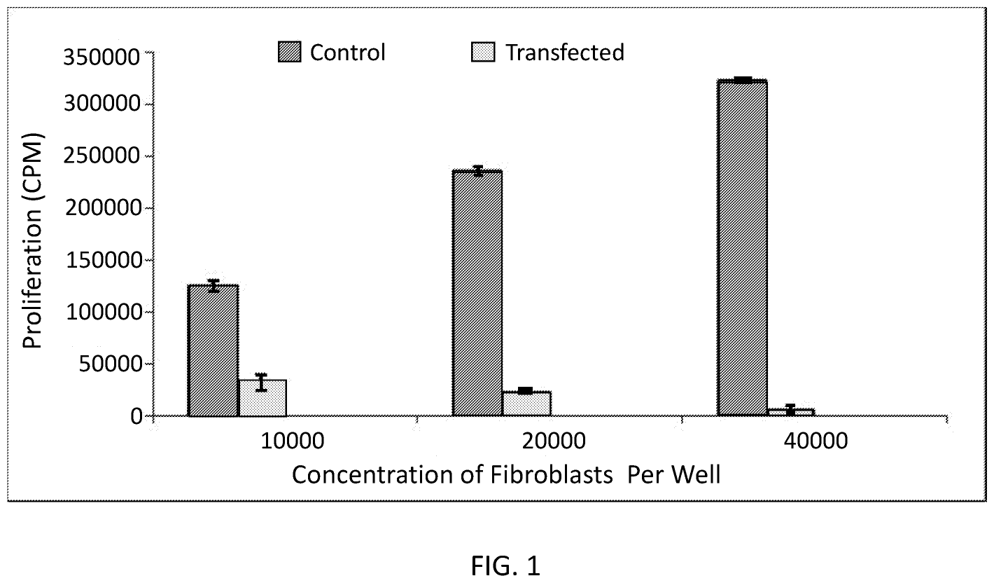

### DETAILED DESCRIPTION

Embodiments of the disclosure generally concern fibroblasts that express one or more chimeric antigen receptors (CAR) that target an antigen and upon binding to the antigen effect a signal through the CAR molecule to induce activity of an endodomain, such as an intracellular signaling domain. Such activity directly or indirectly renders the fibroblasts able to produce one or more compounds that endow the fibroblasts with the ability to induce an immune response, such as a stimulatory immune response.

Disclosed are fibroblast cells (for example, allogeneic, autologous, or xenogenic fibroblasts) useful for the treatment of cancer, including in a universal donor, off-the-shelf manner. In some embodiments of the disclosure, fibroblasts are transfected with a construct encoding at least one CAR that targets an antigen, such as a tumor antigen or a tumor endothelial associated antigen on an antigen binding domain, for example.

In particular embodiments, the CAR-expressing fibroblasts are chemotactic to cancer cells such that upon delivery to an individual are able to home to the cancer cells; they can also migrate to one or more inflammatory sites. Such activities are utilized in certain methods of the disclosure.

### I. Examples of Definitions

In keeping with long-standing patent law convention, the words “a” and “an” when used in the present specification in concert with the word comprising, including the claims, denote “one or more.” Some embodiments of the disclosure may consist of or consist essentially of one or more elements, method steps, and/or methods of the disclosure. It is contemplated that any method or composition described herein can be implemented with respect to any other method or composition described herein.

As used herein, the term “about” or “approximately” refers to a quantity, level, value, number, frequency, percentage, dimension, size, amount, weight or length that varies by as much as 30, 25, 20, 25, 10, 9, 8, 7, 6, 5, 4, 3, 2 or 1% to a reference quantity, level, value, number, frequency, percentage, dimension, size, amount, weight or length. In particular embodiments, the terms “about” or “approximately” when preceding a numerical value indicates the value plus or minus a range of 15%, 10%, 5%, or 1%. With respect to biological systems or processes, the term can mean within an order of magnitude, preferably within 5-fold, and more preferably within 2-fold, of a value. Unless otherwise stated, the term ‘about’ means within an acceptable error range for the particular value.

The term “administered” or “administering”, as used herein, refers to any method of providing a composition to an individual such that the composition has its intended effect on the patient. For example, one method of administering is by an indirect mechanism using a medical device such as, but not limited to a catheter, applicator gun, syringe etc. A second exemplary method of administering is by a direct mechanism such as, local tissue administration, oral ingestion, transdermal patch, topical, inhalation, suppository etc. In some aspects herein, “administering” may be synonymous with “providing”.

As used herein, “allogeneic” refers to tissues or cells from another body that in a natural setting are immunologically incompatible or capable of being immunologically incompatible, although from one or more individuals of the same species.

As used herein, “autologous” refers to tissues or cells that are derived or transferred from the same individual's body (i.e., autologous blood; an autologous bone marrow transplant).

The term “biologically active” refers to any molecule having structural, regulatory or biochemical functions. For example, biological activity may be determined, for example, by restoration of wild-type growth in cells lacking protein activity. Cells lacking protein activity may be produced by many methods (for example, point mutation and frame-shift mutation). Complementation is achieved by transfecting cells that lack protein activity with an expression vector that expresses the protein, a derivative thereof, or a portion thereof. In other cases, a fragment of a gene product (such as a protein) may be considered biologically active (or it may be referred to as functionally active) if it retains the activity of the full-length gene product, although it may be at a reduced but detectable level of the activity of the full-length gene product.

“Cell culture” is an artificial in vitro system containing viable cells, whether quiescent, senescent or (actively) dividing. In a cell culture, cells are grown and maintained at an appropriate temperature, typically a temperature of 37° C. and under an atmosphere typically containing oxygen and CO₂. Culture conditions may vary widely for each cell type though, and variation of conditions for a particular cell type can result in different phenotypes being expressed. The most commonly varied factor in culture systems is the growth medium. Growth media can vary in concentration of nutrients, growth factors, and the presence of other components. The growth factors used to supplement media are often derived from animal blood, such as calf serum.

“Chimeric antigen receptor” or “CAR” or “CARs” as used herein refers to engineered receptors, which graft an antigen specificity onto a cytotoxic cell, for example T cells, NKT cells NK cells and macrophages, and/or onto fibroblast cells. CARs are also known as artificial T-cell receptors, chimeric T-cell receptors or chimeric immunoreceptors. The CARs of the disclosure comprise one, two, or more antigen-specific targeting regions, an extracellular domain (of which the antigen-specific targeting region may be considered to be a part of), a transmembrane domain, one or more co-stimulatory domains (CSD), and one or more intracellular signaling domains (ISD). In some embodiments, the ESD and/or CSD are optional. After the receptor binds specifically to a target antigen, the ISD activates intracellular signaling. For example, the ISD can redirect fibroblast and/or T cell specificity and reactivity toward a selected target in a non-MHC-restricted manner, exploiting the antigen-binding properties of antibodies. The non-MHC-restricted antigen recognition gives cells expressing the CAR the ability to recognize an antigen independent of antigen processing, thus bypassing a major mechanism of tumor escape. In some embodiments, the ISD comprises a protein domain that activates cytokines, including for example, IL-12, IL-7, IL-15, and/or IL-21, or stimulates a T cell and/or NK cell response. In some embodiments the ISD comprises a molecule that inhibits the expression of HLA I and/or HLA II. Moreover, when expressed in T cells, for example, CARs advantageously do not dimerize with endogenous T cell receptor (TCR) alpha and beta chains. In cases where the CAR comprises two or more antigen-specific targeting regions, they may target at least two different antigens and may be arranged in tandem and may or may not be separated by linker sequences. In one embodiment, the CAR is a bispecific CAR. A bispecific CAR is specific to two different antigens.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

The term “co-stimulatory ligand” as used herein includes a molecule on an antigen presenting cell (e.g., dendritic cell, B cell, and the like) that specifically binds a cognate co-stimulatory molecule on a T cell, thereby providing a signal which, in addition to the primary signal provided by, for instance, by the binding of a TCR/CD3 complex with an MHC molecule loaded with peptide, mediates a cellular response, including, but not limited to, proliferation, activation, differentiation, and the like. A co-stimulatory ligand can include, but is not limited to, CD7, B7-1 (CD80), B7-2 (CD86), PD-L1, PD-L2, 4-1BBL, OX40L, an inducible costimulatory ligand (ICOS-L), an intercellular adhesion molecule (ICAM), CD30L, CD40, CD70, CD83, HLA-G, MICA, MICB, HVEM, a lymphotoxin beta receptor, 3/TR6, ILT3, ILT4, HVEM, an agonist or an antibody that binds to a Toll ligand receptor and a ligand that specifically binds with B7-H3. A co-stimulatory ligand also encompasses, inter alia, an antibody that specifically binds with a co-stimulatory molecule present on a cell, such as, but not limited to, CD27, CD28, 4-1BB, OX40, CD30, CD40, PD-1, ICOS, a lymphocyte function-associated antigen-1 (LFA-1), CD2, CD7, LIGHT, NKG2C, B7-H3, and a ligand that specifically binds with CD83.

The term “co-stimulatory molecule” as used herein refers to the cognate binding partner on a T cell that specifically binds with a co-stimulatory ligand, thereby mediating a co-stimulatory response by the T cell, such as, but not limited to, proliferation. Co-stimulatory molecules include, but are not limited to an MHC class 1 molecule, BTLA and a Toll ligand receptor.

The term “co-stimulatory signal” as used herein refers to a signal, which in combination with a primary signal, such as TCR/CD3 ligation, leads to T cell proliferation and/or upregulation or down regulation of key molecules.

The term “cytotoxic cell” or “cytotoxic immune cell” as used herein means a cell which can injure or destroy invading microorganisms, tumor cells or other diseased tissue cells. This term is meant to include natural killer (NK) cells, activated NK cells, neutrophils, T cells, eosinophils, basophils, B-cells, macrophages and lymphokine-activated killer (LAK) cells among other cell types. The cytotoxic cell, through an antibody, receptor, ligand or fragments/derivatives thereof, is bound to a target cell to form a stable complex, and stimulates the cytotoxic cell to destroy the target cell. Cytotoxic cells may also include other immune cells with tumor lytic capabilities including but not limited to natural killer T cells (Heczey et al., “Invariant NKT cells with chimeric antigen receptor provide a novel platform for safe and effective cancer immunotherapy,” Blood, vol. 124, pp. 2824-2833, 2014) and granulocytes. Further, cytotoxic cells may include immune cells with phagocytic capability including but not limited to macrophages and granulocytes, cells with stem cell and/or progenitor cell properties including, but not limited to, hematopoietic stem/progenitor cells (Zhen et al., “HIV-specific Immunity Derived From Chimeric Antigen Receptor-engineered Stem Cells,” Mol Ther., vol. 23, pp. 1358-1367, 2015), embryonic stem cells (ESCs), cord blood stem cells, and induced pluripotent stem cells (iPSCs) (Themeli et al., “New cell sources for T cell engineering and adoptive immunotherapy,” Cell Stem Cell., vol. 16, pp. 357-366, 2015). Additionally, cytotoxic cells include “synthetic cells” such as iPSC-derived T cells (TiPSCs) (Themeli et al., “Generation of tumor-targeted human T lymphocytes from induced pluripotent stem cells for cancer therapy,” Nat Biotechnol., vol. 31, pp. 928-933, 2013) or iPSC-derived NK cells.

“Intracellular signaling domain” (ISD) or “cytoplasmic domain” as used herein refer to the portion of the CAR that transduces the effector function signal and directs the cell to perform its desired function. Examples of domains that transduce the effector function signal include but are not limited to the ζ chain of the T-cell receptor complex or any of its homologs (e.g., η chain, FcsR1γ and β chains, MB 1 (Iga) chain, B29 (Ig) chain, etc.), human CD3 zeta chain, CD3 polypeptides (Δ, δ and ε), syk family tyrosine kinases (Syk, ZAP 70, etc.), src family tyrosine kinases (Lck, Fyn, Lyn, etc.) and other molecules involved in T-cell transduction, such as CD2, CD5 and CD28. In some embodiments, the ISD comprises a protein domain that activates cytokines, including for example, IL-12, IL-7, IL-15, and/or IL-21, or stimulates a T cell and/or NK cell response. In some embodiments the ISD comprises a molecule that inhibits the expression of HLA I and/or HLA II. Other intracellular signaling domains will be apparent to those of skill in the art and may be used in connection with alternate embodiments of the invention.

“Linker” (L) or “linker domain” or “linker region” as used herein refer to an oligo- or polypeptide region from about 1 to 100 amino acids in length, which links together any of the domains/regions of the CAR of the disclosure. Linkers may be composed of flexible residues like glycine and serine so that the adjacent protein domains are free to move relative to one another. Longer linkers may be used when it is desirable to ensure that two adjacent domains do not sterically interfere with one another. Linkers may be cleavable or non-cleavable. Examples of cleavable linkers include 2A linkers (for example T2A), 2A-like linkers or functional equivalents thereof and combinations thereof. In some embodiments, the linkers include the picornaviral 2A-like linker, CHYSEL sequences of porcine teschovirus (P2A), Thosea asigna virus (T2A) or combinations, variants and functional equivalents thereof. In other embodiments, the linker sequences may comprise Asp-Val/Ile-Glu-X-Asn-Pro-Gly1′2A\hat{ }Pro1′2B\hat{ } motif, which results in cleavage between the 2A glycine and the 2B proline. Other linkers will be apparent to those of skill in the art and may be used in connection with alternate embodiments of the invention.

“Mammal” as used herein refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be included within the scope of this term.

“Transmembrane domain” (TMD) as used herein refers to the region of the CAR that crosses the plasma membrane of the fibroblast cell. The transmembrane domain of the CAR of the disclosure may be the transmembrane region of a transmembrane protein (for example Type I transmembrane proteins), an artificial hydrophobic sequence or a combination thereof. Other transmembrane domains will be apparent to those of skill in the art and may be used in connection with alternate embodiments of the disclosure.

The term “drug”, “agent” or “compound” as used herein, refers to any pharmacologically active substance capable of being administered that achieves a desired effect. Drugs or compounds can be synthetic or naturally occurring, non-peptide, proteins or peptides, oligonucleotides, or nucleotides (DNA and/or RNA), polysaccharides or sugars.

The term “individual”, as used herein, refers to a human or animal that may or may not be housed in a medical facility and may be treated as an outpatient of a medical facility. The individual may be receiving one or more medical compositions via the internet. An individual may comprise any age of a human or non-human animal and therefore includes adult, juveniles (i.e., children) and infants. It is not intended that the term “individual” connote a need for medical treatment, therefore, an individual may voluntarily or involuntarily be part of experimentation whether clinical or in support of basic science studies. The term “subject” or “individual” refers to any organism or animal subject that is an object of a method or material, including mammals, e.g., humans, laboratory animals (e.g., primates, rats, mice, rabbits), livestock (e.g., cows, sheep, goats, pigs, turkeys, and chickens), household pets (e.g., dogs, cats, and rodents), horses, and transgenic non-human animals.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

The term “pharmaceutically” or “pharmacologically acceptable”, as used herein, refer to molecular entities and compositions that do not produce adverse, allergic, or other untoward reactions when administered to an animal or a human.

The term, “pharmaceutically acceptable carrier”, as used herein, includes any and all solvents, or a dispersion medium including, but not limited to, water, ethanol, polyol (for example, glycerol, propylene glycol, and liquid polyethylene glycol, and the like), suitable mixtures thereof, and vegetable oils, coatings, isotonic and absorption delaying agents, liposome, commercially available cleansers, and the like. Supplementary bioactive ingredients also can be incorporated into such carriers.

“Therapeutic agent” means to have “therapeutic efficacy” in modulating angiogenesis and/or wound healing and an amount of the therapeutic is said to be a “angiogenic modulatory amount”, if administration of that amount of the therapeutic is sufficient to cause a significant modulation (i.e., increase or decrease) in angiogenic activity when administered to a subject (e.g., an animal model or human patient) needing modulation of angiogenesis.

As used herein, the term “therapeutically effective amount” is synonymous with “effective amount”, “therapeutically effective dose”, and/or “effective dose” and refers to the amount of compound that will elicit the biological, cosmetic or clinical response being sought by the practitioner in an individual in need thereof. As one example, an effective amount is the amount sufficient to reduce immunogenicity of a group of cells. As a non-limiting example, an effective amount is an amount sufficient to promote formation of a blood supply sufficient to support the transplanted tissue. As another non-limiting example, an effective amount is an amount sufficient to promote formation of new blood vessels and associated vasculature (angiogenesis) and/or an amount sufficient to promote repair or remodeling of existing blood vessels and associated vasculature. The appropriate effective amount to be administered for a particular application of the disclosed methods can be determined by those skilled in the art, using the guidance provided herein. For example, an effective amount can be extrapolated from in vitro and in vivo assays as described in the present specification. One skilled in the art will recognize that the condition of the individual can be monitored throughout the course of therapy and that the effective amount of a compound or composition disclosed herein that is administered can be adjusted accordingly.

“Treatment,” “treat,” or “treating” means a method of reducing the effects of a disease or condition. Treatment can also refer to a method of reducing the disease or condition itself rather than just the symptoms. The treatment can be any reduction from pre-treatment levels and can be but is not limited to the complete ablation of the disease, condition, or the symptoms of the disease or condition. Therefore, in the disclosed methods, treatment” can refer to a 10%, 20%, 30%, 40%, 50%, 60%, 70%, 80%, 90%, or 100% reduction in the severity of an established disease or the disease progression, including reduction in the severity of at least one symptom of the disease. For example, a disclosed method for reducing the immunogenicity of cells is considered to be a treatment if there is a detectable reduction in the immunogenicity of cells when compared to pre-treatment levels in the same subject or control subjects. Thus, the reduction can be a 10, 20, 30, 40, 50, 60, 70, 80, 90, 100%, or any amount of reduction in between as compared to native or control levels. It is understood and herein contemplated that “treatment” does not necessarily refer to a cure of the disease or condition, but an improvement in the outlook of a disease or condition. In specific embodiments, treatment refers to the lessening in severity or extent of at least one symptom and may alternatively or in addition refer to a delay in the onset of at least one symptom.

### II. Fibroblast Cells

The disclosure provides genetically engineered fibroblast cells that comprise and stably express one or more CARs encompassed by the disclosure. The CAR expressed by the genetically engineered fibroblast cells may comprise at least one antigen-specific targeting region, at least one transmembrane domain, and at least one intracellular signaling domain. The polynucleotide sequence encoding the CAR may also comprise an N-terminal signal sequence. The antigen-specific targeting domain(s) may be capable of specifically binding, in an MHC unrestricted manner, an antigen that is not normally bound by a T-cell receptor in that manner. The fibroblast cells may or may not also express one or more polypeptides that are not the CAR polypeptide(s).

The genetically engineered fibroblast cells express a CAR that is specific for at least one antigen. In one embodiment, the antigen-specific targeting region comprises target-specific antibodies or functional equivalents or fragments or derivatives thereof. The antigen-specific antibody may be the Fab fragment of the antibody or the single chain variable fragment (scFv) of the antibody, for example.

Genetically modified fibroblast cells may be produced by stably transfecting fibroblast cells with DNA encoding the one or more CARs of the disclosure. DNA encoding the CAR(s) of the disclosure may also encode one or more other polypeptides, including another CAR. In one embodiment, a first polynucleotide encodes the CAR and is linked via IRES sequences or a polynucleotide that encodes a cleavable linker, to a second polynucleotide that encodes another polypeptide. Viral vectors are commonly used to carry heterologous genes into cells (e.g., T-cells) and may or may not be utilized. Examples of viral vectors which may be used to generate genetically modified cells include but are not limited to SIN lentiviral vectors, retroviral vectors, foamy virus vectors, adeno-associated virus (AAV) vectors and/or plasmid transposons (e.g., sleeping beauty transposon system).

Various methods produce stable transfectants that express the CAR(s) of the disclosure. In one embodiment, a method of stably transfecting and re-directing fibroblast cells is by electroporation using naked DNA. By using naked DNA, the time required to produce redirected cells may be significantly reduced. Additional methods to genetically engineer fibroblast cells using naked DNA encoding the CAR of the disclosure include but are not limited to chemical transformation methods (e.g., using calcium phosphate, dendrimers, liposomes and/or cationic polymers), non-chemical transformation methods (e.g., electroporation, optical transformation, gene electrotransfer and/or hydrodynamic delivery) and/or particle-based methods (e.g., impalefection, using a gene gun and/or magnetofection). The transfected fibroblast cells demonstrating presence of a single integrated un-rearranged vector and expression of the CAR may be expanded ex vivo. In one embodiment, the fibroblast cells selected for ex vivo expansion demonstrate the capacity to specifically recognize and lyse antigen-specific target cells. In some embodiments, transfection reagents, such as liposome based transfection reagents, are used to transfect nucleic acids encoding one or more CARs, and optionally other sequences useful for the disclosure, into cells of the disclosure.

The disclosure also provides a method of making and expanding cells expressing one or more CARs. The method comprises transfecting or transducing the fibroblast cells with the vector expressing the CAR and stimulating the cells with cells expressing the target antigens, recombinant target antigens, or an antibody to the receptor to cause the cells to proliferate, so as to make and expand fibroblast cells.

In addition to having the ability to target one or more tumor antigens, and thereby directly or indirectly inhibit tumor growth, the CAR-expressing fibroblasts are capable of stimulating an immune response and thereby have an immune stimulatory phenotype. Such a phenotype allows the CAR-expressing fibroblasts to have the ability to stimulate a mixed lymphocyte reaction. In a mixed lymphocyte reaction, the CAR-expressing fibroblasts are co-cultured with allogeneic lymphocyte cells, proliferation is assessed as a marker of allogenic reactivity. The immune stimulatory phenotype of the CAR-expressing fibroblasts may include the ability to stimulate T cells, NKT cells, and/or NK cells (as compared to fibroblasts that are not CAR-expressing fibroblasts), thereby enhancing an immune response of the individual to inhibit cancer cells of the individual. The CAR-expressing fibroblasts act as antigen presenting cells, in particular embodiments. In specific embodiments, the cells, including NK cells, that are stimulated by the CAR-expressing fibroblasts may or may not be CD94⁺ and/or CD117⁺ and/or CD161⁻ and/or NKG2D⁺ and/or NKp46⁺ and/or CD226⁺ and/or CD57⁺, for example.

In one embodiment of the disclosure, fibroblast cells express at least one CAR capable of endowing the fibroblast with the ability to trigger a T cell-mediated immune response. In one embodiment, the CAR acts as a means of attaching or otherwise bridging fibroblasts to cancer cells. In another embodiment, the CAR acts as a means of triggering enhanced adhesion of the fibroblast to cancer cells. In one specific embodiment, a CAR comprises an extracellular domain capable of binding an antigen, including a tumor or pathogen antigen, and also has an intracellular domain comprising of part or all of the intracellular domain of TLR-4. The intracellular domain of TLR-4 may comprise or consist essentially of or consist of SEQ ID NO:1 (sdfgtts lkyldlsfng vitmssnflg leqlehldfq hsnlkqmsef svflslrnli yldishthtr vafngifngl sslevlkmag nsfqenflpd iftelrnitf ldlsqcqleq lsptafnsls slqvinmshn nffsldtfpy kclnslqvld yslnhimtsk kqelqhfpss laflnitqnd factcehqsf lqwikdqrql lvevermeca tpsdkqgmpv lslnitcqmn ktiigvsvls vlvvsvvavl vykfyfhlml lagcikygrg eniydafviy ssqdedwvrn elvknleegv ppfqlclhyr dfipgvaiaa niihegfhks rkvivvvsqh fiqsrwcife yeiaqtwqfl lqkvektllr qqvelyrlls rntyleweds vlgrhifwrr lrkalldgks wnpegtvgtg cnwqeatsi).

In some embodiments, the fibroblasts comprise activity that may elicit an immune reaction and/or response in an individual. The activity may elicit the CAR-expressing fibroblasts to produce cytokines at a level higher than that in fibroblasts that do not express a CAR. In some embodiments, the CAR-expressing fibroblasts produce cytokines at a level higher than that of fibroblasts that do not express a CAR. Such production of cytokines may elicit an immune reaction and/or response in an individual. The activity may be due to the CAR-expressing fibroblasts The cytokines produced by the fibroblast may include IL-12, IL-7, IL-15, and/or IL-21. The fibroblasts may be manipulated, such as by genetic engineering for example, to produce cytokines, including IL-12, IL-7, IL-15, and/or IL-21. In some embodiments, the CAR comprises a domain that induces the production of cytokines, including IL-12, IL-7, IL-15, and/or IL-21, at a level higher than that of fibroblasts that do not express a CAR. The CAR may or may not be activated, such as by binding to an antigen, to induce the production of cytokines.

CAR-expressing fibroblasts that express increased amounts of at least one anti-apoptotic protein may be administered to an animal in an amount effective to provide a therapeutic effect. The animal may be a mammal, including but not limited to, human and non-human primates.

In certain embodiments, fibroblasts may be derived from tissues comprising skin, heart, blood vessels, bone marrow, skeletal muscle, liver, pancreas, brain, adipose tissue, foreskin, placental, and/or umbilical cord. In specific embodiments, the fibroblasts are placental, fetal, neonatal or adult or mixtures thereof.

In one embodiment of the disclosure, CAR-expressing fibroblasts are transfected with one or more anti-apoptotic proteins to enhance in vivo longevity. The present disclosure includes methods of using CAR-expressing fibroblasts that have been cultured under conditions to express increased amounts of at least one anti-apoptotic protein, for example as a therapy to inhibit or prevent apoptosis. In one embodiment, the CAR-expressing fibroblasts that are used as a therapy to inhibit or prevent apoptosis have been contacted with an apoptotic cell. In specific embodiments, CAR-expressing fibroblasts that have been contacted with an apoptotic cell express high levels of anti-apoptotic molecules. In some instances, the CAR-expressing fibroblasts that have been contacted with an apoptotic cell secrete high levels of at least one anti-apoptotic protein, including but not limited to, STC-1, BCL-2, XIAP, Survivin, Bcl-2XL, GATA-4, FGF-2, and HO-1. In other embodiments, fibroblast cells expressing CAR are transfected to express anti-apoptotic proteins including but not limited to, STC-1, BCL-2, XIAP, Survivin, Bcl-2XL, GATA-4, FGF-2, and HO-1. In addition or alternative to this, in one embodiment of the disclosure, CAR-expressing fibroblasts are pretreated with one or more agents to induce expression of anti-apoptotic genes, and one example is pretreatment with exendin-4.

There are several methods known in the art for the generation of fibroblasts. In one embodiment, fibroblasts are generated according to protocols previously utilized for treatment of patients utilizing bone marrow-derived MSCs. The following example is intended to provide one method for producing fibroblasts, however any method known in the art may be used. Bone marrow is aspirated (for example approximately 10-30 mL of bone marrow) under local anesthesia (with or without sedation) from suitable tissue, such as the posterior iliac crest, collected into tubes, including sodium heparin containing tubes, and transferred to a Good Manufacturing Practices (GMP) clean room. Bone marrow cells are washed with a washing solution such as Dulbecco's phosphate-buffered saline (DPBS), RPMI, or PBS supplemented with autologous patient plasma and layered on to 25 mL of Percoll (1.073 g/mL) at a concentration of approximately 1-2×10⁷ cells/mL. Subsequently the cells are centrifuged at 900 g for approximately 30 min or a time period sufficient to achieve separation of mononuclear cells from debris and erythrocytes. Said cells are then washed with PBS and plated at a density of approximately 1×10⁶ cells per mL in 175 cm² tissue culture flasks in DMEM with 10% FCS with flasks subsequently being loaded with a minimum of 30 million bone marrow mononuclear cells. The fibroblasts are allowed to adhere for 72 h followed by media changes every 3-4 days. Adherent cells are removed with 0.05% trypsin-EDTA and replated at a density of 1×10⁶ per 175 cm². BM-fibroblasts are subsequently transfected with the CAR gene(s).

Fibroblast cell cultures may be tested periodically, such as weekly, for sterility, endotoxin by limulus amebocyte lysate test, and/or mycoplasma by DNA-fluorochrome stain, for example.

In order to determine the quality of fibroblast cultures, flow cytometry may be performed on all cultures for surface expression of fibronectin fibroblast markers and lack of contaminating CD14- and CD-45 positive cells. The following provides an example for determining fibroblast quality, but any method known in the art may be used. Cells were detached with 0.05% trypsin-EDTA, washed with DPBS+2% bovine albumin, fixed in 1% paraformaldehyde, blocked in 10% serum, incubated separately with primary SH-2, SH-3 and SH-4 antibodies followed by PE-conjugated anti-mouse IgG(H+L) antibody. Confluent fibroblasts in 175 cm² flasks are washed with Tyrode's salt solution, incubated with medium 199 (M199) for 60 min, and detached with 0.05% trypsin-EDTA (Gibco). Cells from 10 flasks were detached at a time and fibroblasts were resuspended in 40 mL of M199+1% human serum albumin (HSA; American Red Cross, Washington D.C., USA). fibroblasts harvested from each 10-flask set were stored for up to 4 h at 4° C. and combined at the end of the harvest. A total of 2-10×10⁶ fibroblasts/kg were resuspended in M199+1% HSA and centrifuged at 460 g for 10 min at 20° C. Cell pellets were resuspended in fresh M199+1% HSA media and centrifuged at 460 g for 10 min at 20° C. for three additional times. Total harvest time is 2-4 h based on fibroblast yield per flask and the target dose. Harvested fibroblasts are cryopreserved in Cryocyte (Baxter, Deerfield, Ill., USA) freezing bags using a rate controlled freezer at a final concentration of 10% DMSO (Research Industries, Salt Lake City, Utah, USA) and 5% HSA. On the day of infusion cryopreserved units were thawed at the bedside in a 37° C. water bath and transferred into 60 mL syringes within 5 min and infused intravenously into patients over 10-15 min. Patients are premedicated with 325-650 mg acetaminophen and 12.5-25 mg of diphenhydramine orally. Blood pressure, pulse, respiratory rate, temperature and oxygen saturation are monitored at the time of infusion and every 15 min thereafter for 3 h followed by every 2 h for 6 h.

Based upon the disclosure provided herein, CAR-expressing fibroblasts can be obtained from any source. In particular aspects, the fibroblast cells are isolated from nature or are obtained commercially or are obtained from an individual in need of treatment, or a mixture thereof, for example. The CAR-expressing fibroblasts may be autologous with respect to the recipient (obtained from the same host) or allogeneic with respect to the recipient. In addition, the CAR-expressing fibroblasts may be xenogeneic to the recipient (obtained from an animal of a different species). In one embodiment of the disclosure, CAR-expressing fibroblasts are pretreated with one or more agents to induce expression of anti-apoptotic genes, and one example is pretreatment with exendin-4 (Zhou, H., et al., Exendin-4 protects adipose-derived mesenchymal stem cells from apoptosis induced by hydrogen peroxide through the PI3K/Akt-Sfrp2 pathways. Free Radic Biol Med, 2014. 77: p. 363-75). In a further non-limiting embodiment, CAR-expressing fibroblasts utilized in methods of the present disclosure can be isolated, such as from the bone marrow of any species of mammal, including but not limited to, human, mouse, rat, ape, gibbon, and/or bovine. In a non-limiting embodiment, the CAR-expressing fibroblasts are isolated from a human, a mouse, or a rat. In another non-limiting embodiment, the CAR-expressing fibroblasts are isolated from a human.

Based upon the present disclosure, CAR-expressing fibroblasts can be isolated and expanded in culture in vitro to obtain sufficient numbers of cells for use in the methods described herein provided that the CAR-expressing fibroblasts are cultured in a manner that promotes contact with a tumor cell, such as a tumor endothelial cell. For example, CAR-expressing fibroblasts can be isolated from human bone marrow and cultured in complete medium (DMEM low glucose containing 4 mM L-glutamine, 10% FBS, and 1% penicillin/streptomycin) in hanging drops or on non-adherent dishes. The disclosure, however, should in no way be construed to be limited to any one method of isolating and/or to any culturing medium. Rather, any method of isolating and any culturing medium should be construed to be included in the present disclosure provided that the CAR-expressing fibroblasts are cultured in a manner that provides CAR-expressing fibroblasts that express increased amounts of at least one anti-apoptotic protein.

Any medium capable of supporting fibroblasts, such as CAR-expressing fibroblasts or unmodified fibroblasts, in vitro may be used to culture the CAR-expressing fibroblasts. Media formulations that can support the growth of CAR-expressing fibroblasts include, but are not limited to, Dulbecco's Modified Eagle's Medium (DMEM), alpha modified Minimal Essential Medium (.alpha.MEM), and Roswell Park Memorial Institute Media 1640 (RPMI Media 1640) and the like. The media and conditions for culture of fibroblasts, and by virtue of the disclosure CAR-expressing fibroblasts, are known in the art. Typically, up to 20% fetal bovine serum (FBS) or 1-20% horse serum is added to the above medium in order to support the growth of CAR-expressing fibroblasts. A defined medium, however, also can be used if the growth factors, cytokines, and hormones necessary for culturing CAR-expressing fibroblasts are provided at appropriate concentrations in the medium. Media useful in the methods of the disclosure may comprise one or more compounds of interest, including, but not limited to, antibiotics, mitogenic compounds, or differentiation compounds useful for the culturing of CAR-expressing fibroblasts. The cells may be grown at temperatures between 27° C. to 40° C., such as 31° C. to 37° C., and may be in a humidified incubator. The carbon dioxide content may be maintained between 2% to 10% and the oxygen content may be maintained between 1% and 22%. The disclosure, however, should in no way be construed to be limited to any one method of isolating and culturing CAR-expressing fibroblasts. Rather, any method of isolating and culturing CAR-expressing fibroblasts should be construed to be included in the present disclosure.

Antibiotics that can be added into the medium include, but are not limited to, penicillin and streptomycin. The concentration of penicillin in the culture medium, in a non-limiting embodiment, is about 10 to about 200 units per mL. The concentration of streptomycin in the culture medium is, in a non-limiting embodiment, about 10 to about 200 μg/mL.

The CAR-expressing fibroblasts can be suspended in an appropriate diluent. Suitable excipients for injection solutions are those that are biologically and physiologically compatible with the CAR-expressing fibroblasts and with the recipient, such as buffered saline solution or other suitable excipients. The composition for administration can be formulated, produced, and stored according to standard methods complying with proper sterility and stability. The CAR-expressing fibroblasts may have one or more genes modified or be treated such that the modification has the ability to cause the CAR-expressing fibroblasts to self-destruct “r “commit suic”de” because of such modification, or upon presentation of a second drug (e.g., a prodrug) or signaling compound to initiate such destruction of the CAR-Fibroblasts. In specific embodiments, the CAR-expressing fibroblasts express a suicide gene that may be induced to cause destruction of the cell, such as inducible caspase 9.

The amount of any types of fibroblast cells for administration to an individual may depend on the type of disease to be treated, of the severity and stage of the disease, and/or of the type of cells to be injected for the treatment. The cells may be prepared for administration in a pharmaceutically acceptable carrier, for example a sterile saline isotonic solution. In some embodiments, the pharmaceutically acceptable carrier may comprise one or more additional agents, such as FAS ligand, IL-2R, IL-1 Ra, IL-2, IL-4, IL-8, IL-10, IL-20, IL-35, HLA-G, PD-L1, 1-309, IDO, iNOS, CD200, Galectin 3, sCR1, arginase, PGE-2, aspirin, atorvastatin, fluvastatin, lovastatin, pravastatin, rosuvastatin, simvastatin, pitavastatin, n-acetylcysteine, rapamycin, IVIG, naltrexone, TGF-beta, VEGF, PDGF, CTLA-4, anti-CD45RB antibody, hydroxychloroquine, leflunomide, auranofin, dicyanogold, sulfasalazine, methotrexate, glucocorticoids, etanercept, adalimumab, abatacept, anakinra, certolizumab, Etanercept-szzs, golimumab, infliximab, rituximab, tocilizumab, cyclosporine, IFN-gamma, everolimus, rapamycin, VEGF, FGF-1, FGF-2, angiopoietin, HIF-1-alpha, or a combination thereof.

In cases wherein recombination technology is employed, one or more types of fibroblast cells are manipulated to harbor an expression vector that encodes a gene product of interest (that may or may not be a CAR). A recombinant expression vector(s) can be introduced as one or more DNA molecules or constructs, where there may be at least one marker that will allow for selection of host cells that contain the vector(s). The vector(s) can be prepared in conventional ways, wherein the genes and regulatory regions may be isolated, as appropriate, ligated, cloned in an appropriate cloning host, and analyzed by sequencing or other convenient means. Particularly, using PCR, individual fragments including all or portions of a functional unit may be isolated, where in some cases one or more mutations may be introduced usi“g “primer rep”ir”, ligation, in vitro mutagenesis, etc. as appropriate. The vector(s) once completed and demonstrated to have the appropriate sequences may then be introduced into the host cell by any convenient means. The constructs may be integrated and packaged into non-replicating, defective viral genomes like lentivirus, Adenovirus, Adeno-associated virus (AAV), Herpes simplex virus (HSV), or others, including retroviral vectors, for infection or transduction into cells. The vector(s) may include viral sequences for transfection, if desired. Alternatively, the construct may be introduced by fusion, electroporation, biolistics, transfection, lipofection, or the like. The host cells may be grown and expanded in culture before introduction of the vector(s), followed by the appropriate treatment for introduction of the vector(s) and integration of the vector(s). The cells are then expanded and screened by virtue of a marker present in the construct. Various markers that may be used successfully include hprt, neomycin resistance, thymidine kinase, hygromycin resistance, etc.

Any of the genes or gene products described herein, or active portions thereof, may be cloned into mammalian expression constructs comprising one or more promoter sequences enabling expression in cells such as the CMV promoter [Artuc et al., Exp. Dermatol. 1995, 4:317-21]. Examples of suitable constructs include, but are not limited to pcDNA3, pcDNA3.1 (+/−), pGL3, PzeoSV2 (+/−), pDisplay, pEF/myc/cyto, pCMV/myc/cyto each of which is commercially available from Invitrogen Co. (www.invitrogen.com), or the pSH expression vector which enables a regulated polynucleotide expression in human foreskin cells [Ventura and Villa, 1993, Biochem. Biophys. Commun. 192: 867-9]. Examples of retroviral vector and packaging systems are those sold by Clontech, San Diego, Calif., USA, including Retro-X vectors pLNCX and pLXSN, which permit cloning into multiple cloning sites and the transgene is transcribed from CMV promoter. Vectors derived from Mo-MuLV are also included such as pBabe, where the transgene will be transcribed from th′ 5′LTR promoter. After completing plasmid transfection fibroblasts are harvested by a means allowing for detachment from tissue culture plates, for example, by trypsinization and transferred to either a 6-well (Nunc, Denmark) or a 24-well plate (Nunc) for proliferation. Approximately 3 days post-transfection, the cell media is changed to media allow for proliferation and expansion of modified fibroblasts. One example is Neurobasal A (NBA) proliferation medium comprising Neurobasal-A (Invitrogen), 1% D-glucose (Sigma Aldrich), 1% Penicillin/Streptomycin/Glutamine (Invitrogen), 2% B27 supplement with Retinoic acid (Invitrogen), 0.2% EGF (Peprotech, USA), 0.08% FGF-2 (Peprotech), 0.2% Heparin (Sigma Aldrich, USA) and Valproic acid (Sigma-Aldrich) to a concentration of 1 μM. The media is then subsequently changed thrice weekly, and cells are re-plated regularly (for example, 2-8 times up to a maximum of weekly re-plating, becoming more regular as colonies began to develop) to remove non-reprogrammed cells until widespread colony formation is achieved.

In some instances, one or more agents, such as angiogenic agents or functional fragments thereof, may be introduced into the cells as an RNA molecule for transient expression. RNA can be delivered to any cells, including any modified cells, of the disclosure by various means including microinjection, electroporation, and lipid-mediated transfection, for example. In particular aspects, introduction of vector(s) into cells may occur via transposons. An example of a synthetic transposon for use is the Sleeping Beauty transposon that comprises an expression cassette including the angiogenic agent gene thereof. Alternatively, one may have a target site for homologous recombination, where it is desired that vector(s) be integrated at a particular locus using materials and methods as are known in the art for homologous recombination. For homologous recombination, one may use either OMEGA or O-vectors. See, for example, Thomas and Capecchi, 1987; Mansour, et al., 1988; and Joyner, et al., 1989.

The vector(s) may be introduced as a single DNA molecule encoding at least one agent (including one or more angiogenic agent or functional fragments thereof) and optionally another polynucleotide (such as genes), or different DNA molecules having one or more polynucleotides (such as genes). The vector(s) may be introduced simultaneously or consecutively, each with the same or different markers. In an illustrative example, one vector would contain one or more agents (such as angiogenic agent(s)) under the control of particular regulatory sequences.

Vector(s) comprising useful elements such as bacterial or yeast origins of replication, selectable and/or amplifiable markers, promoter/enhancer elements for expression in prokaryotes or eukaryotes, etc. that may be used to prepare stocks of vector DNAs and for carrying out transfections are well known in the art, and many are commercially available.

In certain embodiments, it is contemplated that RNAs or proteinaceous sequences may be co-expressed with other selected RNAs or proteinaceous sequences in the same host cell. Co-expression may be achieved by co-transfecting the host cell with two or more distinct recombinant vectors. Alternatively, a single recombinant vector may be constructed to include multiple distinct coding regions for RNAs, which could then be expressed in host cells transfected with the single vector.

In some situations, it may be desirable to kill the modified cells, such as when the object is to terminate the treatment (for example, because of success or lack thereof), the cells become neoplastic, in research where the absence of the cells after their presence is of interest, and/or another event. For this purpose one can provide for the expression of certain gene products in which one can kill the modified cells under controlled conditions, such as a suicide gene. Suicide genes are known in the art, e.g. the iCaspase9 system in which a modified form of caspase 9 is dimerizable with a small molecule, e.g. AP1903. See, e.g., Straathof et al., Blood 105:4247-4254 (2005).

### III. Chimeric Antigen Receptors

In particular aspects, chimeric antigen receptors (CARs) are utilized in fibroblast cells for a therapeutic purpose and are comprised of multiple components that are not similarly grouped in a natural setting. The CARs are synthetic and made by the hand of man. Included in the scope of the disclosure are functional portions of the inventive CARs described herein. In one embodiment the CAR is utilized to activate fibroblast cells, for example to endow cytokine production from the fibroblasts and/or to stimulate cytotoxicity against tumors or cancer cells of any kind.

In specific embodiments, the intracellular domain of the CAR comprises at least CD3 zeta chain. In addition to this, or as an alternative, the CAR may comprise one or more intracellular domains, and the intracellular domains may or may not have the activity of being costimulatory domains. As one example, the intracellular domain may comprise an activator of one or more molecular pathways that endows an immune stimulatory phenotype to the fibroblast, such as an intracellular domain of the TLR-4 protein and/or at least one shRNA domain encoding a transcript that generates at least one siRNA capable of inhibiting expression of HLA I and/or HLA II (including the HLA genes that are non-polymorphic). The fibroblasts are engineered in a manner such that binding of the CAR to its cognate ligand results in transduction of a signal stimulating release of one or more tumor cytotoxic factors, such as TRAIL, TNF-alpha, or TNF-beta, for example. In some embodiments, such CAR-expressing fibroblasts are generated to secrete one or more immune stimulatory cytokines, such as IL-12, IL-15 or IL-21. When the intracellular domain is a costimulatory domain, the costimulatory domain may be any one or more of, for example, members of the TNFR superfamily, CD40L, CD28, CD137 (4-1BB), CD134 (OX40), DAP10, DAP12, CD27, CD2, CD5, ICAM-1, LFA-1 (CD11a/CD18), Lck, TNFR-I, TNFR-II, Fas, CD30, CD40 or combinations thereof, for example.

The CARs encompassed herein may be synthesized as single polypeptide chains and may comprise at least one antigen-specific targeting region, optionally an extracellular spacer domain, a transmembrane domain, and one or more intracellular signaling domains. In one embodiment, the antigen-specific targeting region(s) are at the N-terminus, and if the CAR is bispecific they may be arranged in tandem and may be separated by a linker peptide. The antigen-specific targeting region optionally may be linked to an extracellular spacer domain which is linked to the transmembrane domain. The transmembrane domain may be linked to an intracellular signaling domain that may be at the C-terminus. Polynucleotides encoding these polypeptides may further comprise an N-terminal signal sequence that directs the CAR to the cell surface as a type I transmembrane protein. The antigen-specific targeting region may be extracellular-facing and the intracellular signaling domain may be cytoplasmic.

In cases where the CAR is a bispecific CAR, the antigens targeted by the bispecific CAR may be antigens on a single diseased cell or antigens that are expressed on separate cells that each contribute to the disease. The antigen(s) targeted by the CAR are antigen(s) that are either directly or indirectly involved in the disease. In a bispecific CAR, at least two different antigen-specific antibodies or fragments thereof or derivatives thereof may be cloned into the antigen-specific targeting region. The antibodies may be specific for any, but at least two, distinct antigens of choice. The antibody specific to the antigen may be the Fab fragment of the antibody or the single chain variable fragment (scFv) of the antibody.

In one embodiment, CAR-transfected fibroblasts are generated with one or more CARs comprising an antigen binding domain that is capable of binding to an antigen, such as a tumor antigen.

In one embodiment of the disclosure the ability of fibroblasts to inhibit cancer cell growth is amplified by transfection with one or more CARs, wherein the CAR intracellular domain activates one or more tumoricidal genes, such as TRAIL, TNF, Type 1 or Type 2 interferons, for example. Means of transfecting cells similar to fibroblasts with tumoricidal, tumor inhibitory, or immune stimulatory genes would be routine in the art.

The definition of “functional portion” when used in reference to a particular CAR refers to any part or fragment of the CAR of the disclosure, which part or fragment retains the biological activity of the CAR of which it is a part (the parent CAR). Functional portions encompass, for example, those parts of a CAR that retain the ability to recognize target cells, or detect, treat, or prevent a disease, to a similar extent, the same extent, or to a higher extent, as the parent CAR. In reference to the parent CAR, the functional portion can comprise, for instance, about 10%, 25%, 30%, 50%, 60%, 70%, 80%, 90%, 95%, or more, of the parent CAR. The functional portion can comprise additional amino acid(s) at the amino or carboxy terminus of the portion, or at both termini, which additional amino acids are not found in the amino acid sequence of the parent CAR. Desirably, the additional amino acids do not interfere with the biological function of the functional portion, e.g., recognize target cells, detect cancer, treat or prevent cancer. A functional variant can, for example, comprise the amino acid sequence of the parent CAR with at least one conservative amino acid substitution. Alternatively or additionally, the functional variants can comprise the amino acid sequence of the parent CAR with at least one non-conservative amino acid substitution. In this case, it is preferable for the non-conservative amino acid substitution to not interfere with or inhibit the biological activity of the functional variant. The non-conservative amino acid substitution may enhance the biological activity of the functional variant, such that the biological activity of the functional variant is increased as compared to the parent CAR. Amino acid substitutions of the inventive CARs are preferably conservative amino acid substitutions. Conservative amino acid substitutions are known in the art, and include amino acid substitutions in which one amino acid having certain physical and/or chemical properties is exchanged for another amino acid that has the same or similar chemical or physical properties. For instance, the conservative amino acid substitution can be an acidic/negatively charged polar amino acid substituted for another acidic/negatively charged polar amino acid (e.g., Asp or Glu), an amino acid with a nonpolar side chain substituted for another amino acid with a nonpolar side chain (e.g., Ala, Gly, Val, Ile, Leu, Met, Phe, Pro, Trp, Cys, Val, etc.), a basic/positively charged polar amino acid substituted for another basic/positively charged polar amino acid (e.g. Lys, His, Arg, etc.), an uncharged amino acid with a polar side chain substituted for another uncharged amino acid with a polar side chain (e.g., Asn, Gln, Ser, Thr, Tyr, etc.), an amino acid with a beta-branched side-chain substituted for another amino acid with a beta-branched side-chain (e.g., Ile, Thr, and Val), an amino acid with an aromatic side-chain substituted for another amino acid with an aromatic side chain (e.g., His, Phe, Trp, and Tyr), etc.

The CAR can comprise, consist of, or consist essentially of the specified amino acid sequence or sequences described herein, such that other components, e.g., other amino acids, do not materially change the biological activity of the functional variant. The CARs of the disclosure retain their biological activity, e.g., the ability to specifically bind to antigen, detect diseased cells in a mammal, or treat or prevent disease in a mammal, etc. For example, the CAR can be about 50 to about 5000 amino acids long, such as 50, 70, 75, 100, 125, 150, 175, 200, 300, 400, 500, 600, 700, 800, 900, 1000 or more amino acids in length.

CARs of embodiments of the disclosure (including functional portions and functional variants of the disclosure) can comprise synthetic amino acids in place of one or more naturally-occurring amino acids. Such synthetic amino acids are known in the art, and include, for example, aminocyclohexane carboxylic acid, norleucine, .alpha.-amino n-decanoic acid, homoserine, S-acetylaminomethyl-cysteine, trans-3- and trans-4-hydroxyproline, 4-aminophenylalanine, 4-nitrophenylalanine, 4-chlorophenylalanine, 4-carboxyphenylalanine, .beta.-phenylserine .beta.-hydroxyphenylalanine, phenylglycine, .alpha.-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide′ N′-benzy′-N′-methyl-lysine′ N′,N′-dibenzyl-lysine, 6-hydroxylysine, ornithine, alpha-aminocyclopentane carboxylic acid, alpha-aminocyclohexane carboxylic acid, alpha-aminocycloheptane carboxylic acid, alpha-(2-amino-2-norbornane)-carboxylic acid, alpha,gamma-diaminobutyric acid, alpha,beta-diaminopropionic acid, homophenylalanine, and alpha-tert-butylglycine. The CARs of embodiments of the disclosure (including functional portions and functional variants) can be glycosylated, amidated, carboxylated, phosphorylated, esterified, N-acylated, cyclized via, e.g., a disulfide bridge, or converted into an acid addition salt and/or optionally dimerized or polymerized, or conjugated.

The CARs of embodiments of the disclosure (including functional portions and functional variants thereof) can be obtained or generated by methods known in the art. The CARs may be made by any suitable method of making polypeptides or proteins. Suitable methods of de novo synthesizing polypeptides and proteins are described in references, such as Chan et al., Fmoc Solid Phase Peptide Synthesis, Oxford University Press, Oxford, United Kingdom, 2000; Peptide and Protein Drug Analysis, ed. Reid, R., Marcel Dekker, Inc., 2000; Epitope Mapping, ed. Westwood et al., Oxford University Press, Oxford, United Kingdom, 2001; and U.S. Pat. No. 5,449,752. Also, polypeptides and proteins can be recombinantly produced using the nucleic acids described herein using standard recombinant methods. See, for instance, Sambrook et al., Molecular Cloning: A Laboratory Manual, 3^{rd} ed., Cold Spring Harbor Press, Cold Spring Harbor, N.Y. 2001; and Ausubel et al., Current Protocols in Molecular Biology, Greene Publishing Associates and John Wiley & Sons, N Y, 1994. Further, some of the CARs of the invention (including functional portions and functional variants thereof) can be isolated and/or purified from a source, such as a plant, a bacterium, an insect, a mammal, e.g., a rat, a human, etc. Methods of isolation and purification are well-known in the art. Alternatively, the CARs described herein (including functional portions and functional variants thereof) can be commercially synthesized by companies, such as Synpep (Dublin, Calif.), Peptide Technologies Corp. (Gaithersburg, Md.), and Multiple Peptide Systems (San Diego, Calif.). In this respect, the inventive CARs can be synthetic, recombinant, isolated, and/or purified.

A. Antigen-Specific Targeting Region

In particular embodiments the CAR molecule comprises at least one antigen-specific targeting region that targets an antigen, such as an antigen on a diseased cell. The antigen-specific targeting region may target a tumor antigen or a pathogen antigen, for example.

In specific embodiments, the antigen-specific targeting region comprises an antibody, or antigen binding portion thereof. The antibody can be any type of immunoglobulin that is known in the art. For instance, the antibody can be of any isotype, e.g., IgA, IgD, IgE, IgG, IgM, etc. The antibody can be monoclonal or polyclonal. The antibody can be a naturally-occurring antibody, e.g., an antibody isolated and/or purified from a mammal, e.g., mouse, rabbit, goat, horse, chicken, hamster, human, etc. Alternatively, the antibody can be a genetically-engineered antibody, e.g., a humanized antibody or a chimeric antibody. The antibody can be in monomeric or polymeric form. Also, the antibody can have any level of affinity or avidity for the functional portion of the inventive CAR. In specific embodiments, antibodies that bind to a CAR encompassed by the disclosure are utilized to render the CAR-T cells ineffective, such as when they are no longer needed.

In one embodiment, an antigen-specific targeting region comprises the full-length IgG heavy chain (specific for the target antigen) having the VH, CHI, hinge, and the CH2 and CH3 (Fc) Ig domains, if the VH domain alone is sufficient to confer antigen-specificit “(“single-domain antibod”es”). The full length IgG heavy chain may be linked to the co-stimulatory domain and the intracellular signaling domain via the appropriate transmembrane domain. If both the VH and the VL domains are necessary to generate a fully active antigen-specific targeting region, the VH-containing CAR and the full-length lambda light chain (IgL) may both be introduced into the fibroblast cells to generate an active antigen-specific targeting region. In an embodiment, an extracelluar spacer domain may be linked between the antigen-specific binding domain and the transmembrane domain.

In a certain embodiment, an antigen-specific targeting region of the CAR comprises a single chain antibody variable fragment (scFv) specific for a target antigen (and bispecific CARs comprise two scFvs specific for non-identical target antigens). scFvs, in which the C-terminus of one variable domain (VH or VL) is tethered to the N-terminus of the other (VL or VH, respectively) via a polypeptide linker, have been developed without significantly disrupting antigen binding or specificity of the binding. (Chaudhary et al., A recombinant single-chain immunotoxin composed of anti-Tac variable regions and a truncated diphtheria toxin. 1990 Proc. Natl. Acad. Sci., 87:9491; Bedzyk et al. Immunological and structural characterization of a high affinity anti-fluorescein single-chain antibody. 1990 J. Biol. Chem., 265: 18615). The linker connects the N-terminus of the VH with the C-terminus of VL or the C-terminus of VH with the N-terminus of VL. These scFvs lack the constant regions (Fc) present in the heavy and light chains of the native antibody. The scFvs are arranged linked to the intracellular signaling domain via a transmembrane domain. In an embodiment, an extracellular spacer domain may be linked between the antigen-specific binding region and the transmembrane domain.

In another aspect, a scFv fragment may be fused to all or a portion of the constant domains of the heavy chain. The resulting antigen-specific targeting region, specific for one or at least two different antigens, is joined to the co-stimulatory domain and the intracellular signaling domain via a transmembrane domain. In an embodiment, an extracelluar spacer domain may be linked between the antigen-specific binding domain and the transmembrane domain.

In a further embodiment, an antigen-specific targeting region of the CAR comprises a divalent (or bivalent) single-chain variable fragment (di-scFv, bi-scFv). In bispecific CARs comprising di-scFVs, two scFvs specific for each antigen are linked together by producing a single peptide chain with two VH and two VL regions, yielding tandem scFvs. (Xiong, Cheng-Yi; Natarajan, A; Shi, X B; Denardo, G L; Denardo, S J (2006“. “Development of tumor targeting anti-MUC-1 multimer: effects of di-scFv unpaired cysteine location on PEGylation and tumor bind”ng”. Protein Engineering Design and Selection 19 (8): 359-367; Kufer, Peter; Lutterbuse, Ralf; Baeuerle, Patrick A. (2004“. “A revival of bispecific antibod”es”. Trends in Biotechnology 22 (5): 238-244). CARs comprising at least two antigen-specific targeting regions would express two scFvs specific for each of the two antigens. The resulting antigen-specific targeting region, specific for at least two different antigens, is joined to the co-stimulatory domain and the intracellular signaling domain via a transmembrane domain. In an embodiment, an extracelluar spacer domain may be linked between the antigen-specific binding domain and the transmembrane domain.

In an additional embodiment, an antigen-specific targeting region of the CAR comprises a diabody. In a diabody, the scFvs are created with linker peptides that are too short for the two variable regions to fold together, driving the scFvs to dimerize. Still shorter linkers (one or two amino acids) lead to the formation of trimers, the so-called triabodies or tribodies. Tetrabodies may also be used.

To generate bispecific CARs, two or more individual antigen-specific targeting regions are connected to each other, either covalently or noncovalently, on a single protein molecule. An oligopeptide or polypeptide linker, an Fc hinge or membrane hinge region may be used to connect these domains to each other. In bispecific CARs, they may comprise two or more of the different antigen-specific targeting regions connected together in different combinations. For example, two or more antigen-specific targeting regions containing immunoglobulin sequences (e.g. scFvs and/or single-domain antibodies) may be linked to each other.

Targets of antigen-specific targeting regions of CARs may be of any kind. In some embodiments, the antigen-specific targeting region of the CAR targets antigens specific for cancer, inflammatory disease, neuronal-disorders, diabetes, cardiovascular disease, infectious diseases or a combination thereof. Examples of antigens that may be targeted by the CARs include but are not limited to antigens expressed on B-cells, antigens expressed on carcinomas, sarcomas, lymphomas, leukemia, germ cell tumors, blastomas, antigens expressed on various immune cells, and antigens expressed on cells associated with various hematologic diseases, autoimmune diseases, and/or inflammatory diseases. The CARs of the disclosure may be capable of redirecting the effector function of the expressing-cells to the target antigen(s).

In particular embodiments, the CAR(s) of the CAR-expressing fibroblasts have affinity for a particular tumor antigen. In an exemplary embodiment, the genetically engineered cells of the disclosure express a CAR that is specific for TEM-1, TEM-2, TEM-3, TEM-4, TEM-5, TEM-6, TEM-7, TEM-8, ROBO-4, VEGRF2, CD1032urvivingvin and/or CD93 antigen(s).

Embodiments of the disclosure include fibroblast cells that express a chimeric antigen receptor (CAR) comprising: a) at least one extracellular antigen-specific targeting region; b) at least one transmembrane domain; and c) at least one intracellular signaling domain. In specific embodiments, the antigen-specific targeting region targets a cancer antigen, including a tumor antigen.

Antigens that may be targeted by the CARs of the disclosure include but are not limited to any one or more of 4-IBB, 707-AP, 5T4, adenocarcinoma antigen, alpha-fetoprotein, BAFF, B-lymphoma cell, C242 antigen, CA-125, carbonic anhydrase 9 (CA-IX), C-MET, ART-4, BAGE, b-catenin/m, bcr-abl, CAMEL, CAP-1, CCR4, CD 152, CD7, CD 19, CD20, CD200, CD22, CD221, CD23 (IgE receptor), CD28, CD30 (TNFRSF8), CD33, CD4, CD38, CD40, CD44 v6, CD44v7/8, CD51, CD52, CD56, CD74, CD80, CD93, CD123, CD171, CEA, CLPP, CNT0888, CTLA-4, carcinoembryonic antigen, EGP2, EGP40, DR5, ErbB2, ErbB3/4, EGFR, EpCAM, EPV-E6, CD3, CASP-8, CD109, CDK/4, CDC-27, Cyp-B, DAM-8, DAM-10, ELV-M2, ETV6, FAP, fibronectin extra domain-B, folate receptor 1, GAGE, GD2, GD3 ganglioside, glycoprotein 75, GPNMB, G250, Gp100, HAGE, HER2/neu, HGF, HMW-MAA, human scatter factor receptor kinase, hTERT, IGF-1 receptor, IGF-I, IgG1, -I-CAM, IL-13, IL-6, insulin-like growth factor I receptor, integrin α5β1, integrin αvβ3, Kappa or light chain, LAGE, Lewis Y, G250/CAIX, Glypican-3, MAGE, MC1-R, mesothelin, MORAb-009, MS4A1, MUC1, MUC16, mucin CanAg, N-glycolylneuraminic acid, NPC-1C, PDGF-R a, PDL192, phosphatidylserine, PSC1, PSMA, NKG2D ligands, RANKL, RON, ROR1, SAGE, SCH 900105, SDC1, SLAMF7, TAG-72, TEL/AML, tenascin C, TGF beta 2, TGF-β, TRAIL-R1, TRAIL-R2, tumor antigen CTAA16.88, VEGF-A, VEGFR-1, VEGFR2, vimentin, B7-H6, IL-13 receptor a2, IL-11 receptor Ra, 8H9, NCAM, Fetal AchR, iCE, MART-1, tyrosinase, WT-1, TEM-1, TEM-2, TEM-3, TEM-4, TEM-5, TEM-6, TEM-7, TEM-8, ROBO-4, and so forth. Other antigens specific for cancer will be apparent to those of skill in the art and may be used in connection with alternate embodiments of the invention.

Particular examples of target antigens include but are not limited to surface proteins found on cancer cells in a specific or amplified fashion (e.g. the IL-14 receptor, CD 19, CD20 and CD40 for B-cell lymphoma, the Lewis Y and CEA antigens for a variety of carcinomas, the Tag72 antigen for breast and colorectal cancer, EGF-R for lung cancer, folate binding protein and the HER-2 protein that is often amplified in human breast and ovarian carcinomas), or viral proteins (e.g. gp120 and gp41 envelope proteins of HIV, envelope proteins from the Hepatitis B and C viruses, the glycoprotein B and other envelope glycoproteins of human cytomegalovirus, the envelope proteins from oncoviruses such as Kap′si's sarcoma-associated Herpes virus). Other targets of the CARs of the disclosure include CD4, where the ligand is the HIV gp120 envelope glycoprotein, and other viral receptors, for example ICAM, which is the receptor for the human rhinovirus, and the related receptor molecule for poliovirus.

In some embodiments, the bispecific chimeric antigen receptors target and bind at least two different antigens. Examples of pairings of at least two antigens bound by the bispecific CARs of the disclosure include but are not limited to any combination with HER2, CD 19 and CD20, CD 19 and CD22, CD20 and -I-CAM, -I-CAM and GD2, EGFR and -I-CAM, EGFR and C-MET, EGFR and HER2, C-MET and HER2 and EGFR and ROR1. Other pairings of antigens specific for cancer will be apparent to those of skill in the art and may be used in connection with alternate embodiments of the disclosure. In yet other embodiments, the bispecific chimeric antigen receptor targets CD 19 and CD20.

Antigens specific for inflammatory diseases that may be targeted by the CARs of the disclosure include but are not limited to any one or more of AOC3 (VAP-1), CAM-3001, CCL11 (eotaxin-1), CD125, CD147 (basigin), CD154 (CD40L), CD2, CD20, CD23 (IgE receptor), CD25 (a chain of IL-2 receptor), CD3, CD4, CD5, IFN-a, IFN-γ, IgE, IgE Fc region, IL-1, IL-12, IL-23, IL-13, IL-17, IL-17A, IL-22, IL-4, IL-5, IL-5, IL-6, IL-6 receptor, integrin a4, integrin α4β7, Lama glama, LFA-1 (CD11a), MEDI-528, myostatin, OX-40, rhuMAb (37, scleroscin, SOST, TGF beta 1, TNF-α or VEGF-A. Other antigens specific for inflammatory diseases will be apparent to those of skill in the art and may be used in connection with alternate embodiments of the invention.

Antigens specific for neuronal disorders that may be targeted by the CARs of the disclosure include but are not limited to any one or more of beta amyloid or MABT5102A. Other antigens specific for neuronal disorders will be apparent to those of skill in the art and may be used in connection with alternate embodiments of the invention.

Antigens specific for diabetes that may be targeted by the CARs of the disclosure include but are not limited to any one or more of L-43 or CD3. Other antigens specific for diabetes or other metabolic disorders will be apparent to those of skill in the art and may be used in connection with alternate embodiments of the invention.

Antigens specific for cardiovascular diseases which may be targeted by the CARs of the disclosure include but are not limited to any one or more of C5, cardiac myosin, CD41 (integrin alpha-lib), fibrin II, beta chain, ITGB2 (CD 18) and sphingosine-1-phosphate. Other antigens specific for cardiovascular diseases will be apparent to those of skill in the art and may be used in connection with alternate embodiments of the disclosure.

Antigens specific for infectious diseases that may be targeted by the CARs of the disclosure include but are not limited to any one or more of anthrax toxin, CCR5, CD4, clumping factor A, cytomegalovirus, cytomegalovirus glycoprotein B, endotoxin, Escherichia coli, hepatitis B surface antigen, hepatitis B virus, HIV-1, Hsp90, Influenza A hemagglutinin, lipoteichoic acid, Pseudomonas aeruginosa, rabies virus glycoprotein, respiratory syncytial virus and TNF-a. Other antigens specific for infectious diseases will be apparent to those of skill in the art and may be used in connection with alternate embodiments of the disclosure.

Additional targets of the CARs of the disclosure include antigens involved in B-cell associated diseases. Yet further targets of the CARs of the disclosure will be apparent to those of skill in the art and may be used in connection with alternate embodiments of the invention.

B. Transmembrane Domain

The CARs of the disclosure may also comprise a transmembrane domain. The transmembrane domain may comprise the transmembrane sequence from any protein that has a transmembrane domain, including any of the type I, type II or type III transmembrane proteins. The transmembrane domain of the CAR of the disclosure may also comprise an artificial hydrophobic sequence. The transmembrane domains of the CARs of the disclosure may be selected so as not to dimerize. Additional transmembrane domains will be apparent to those of skill in the art and may be used in connection with alternate embodiments of the disclosure.

In particular embodiments, the CAR comprises a transmembrane domain selected from the group consisting of CD3-zeta, CD28, CD8a, CD4, or a combination thereof, for example.

C. Intracellular Signaling Domain

The intracellular signaling domain may be of any kind, but in specific embodiments it is linked to an activator of a molecular pathway that endows an immune stimulatory phenotype, such as having enhanced ability to stimulate a mixed lymphocyte reaction (mixing lymphocytes with the fibroblasts and seeing if lymphocytes proliferate or produce cytokines in response) as compared to a naïve fibroblast. For example, the immune stimulatory phenotype may be an enhanced ability to inhibit tumor growth as compared to a naïve fibroblast or a fibroblast that does not express a CAR. The immune stimulatory phenotype may be an enhanced ability to stimulate NK cells as compared to a naïve fibroblast. The immune stimulatory phenotype may be an enhanced ability to stimulate a T cell response as compared to a naïve fibroblast, such as a T cell response that is Th1. The NK cells may or may not be CD94⁺ and/or CD117⁺ and/or CD161⁻ and/or NKG2D⁺ and/or NKp46⁺ and/or CD226⁺ and/or CD57⁺, for example.

In particular embodiments, the intracellular signaling domain is linked to one or more activators of a molecular pathway that endows an immune stimulatory phenotype and in specific embodiments the activator of molecular pathways that endows an immune stimulatory phenotype is an intracellular domain of the TLR-4 protein and/or at least one shRNA domain encoding a transcript that generates at least one siRNA capable of inhibiting expression of HLA I and/or HLA II. Various common sequences of the HLA molecule may be targeted. In one embodiment, the CLIP protein, which is involved in processing and maturation of HLA may be silenced in order to block expression of HLA molecules.

In some embodiments, the intracellular signaling domain comprises a protein domain that activates cytokines, including for example, IL-12, IL-7, IL-15, and/or IL-21, or stimulates a T cell and/or NK cell response. Examples include JAK (Janus activating kinase) and ITAMs (Immune tyrosine activating motifs) protein domains.

In particular aspects, the activator of molecular pathways that endow an immune stimulatory phenotype is the functional portion of the TLR-4 protein (for example, SEQ ID NO:1) that interacts with MyD88 at a sufficient affinity to trigger MyD88 signal transduction. In specific cases, the activator is the functional portion of the TLR-4 protein that interacts with TRAM and MAL at a sufficient affinity (for example, as seen with phosphorylation assays) to trigger TLR4 signal transduction.

In specific embodiments, an intracellular signaling domain transduces the effector function signal and directs the cell to perform its specialized function. Examples of intracellular signaling domains include, but are not limited to, ζ chain of the T-cell receptor or any of its homologs {e.g., η chain, FcsR1γ and β chains, MB1 (Iga) chain, B29 (Ig) chain, etc.), CD3 polypeptides (A, 6 and c), syk family tyrosine kinases (Syk, ZAP 70, etc.), src family tyrosine kinases (Lck, Fyn, Lyn, etc.) and other molecules involved in T-cell transduction, such as CD2, CD5 and CD28. Specifically, the intracellular signaling domain may be human CD3 zeta chain, FcγRIII, FcsRI, cytoplasmic tails of Fc receptors, immunoreceptor tyrosine-based activation motif (ITAM) bearing cytoplasmic receptors or combinations thereof. Additional intracellular signaling domains will be apparent to those of skill in the art and may be used in connection with alternate embodiments of the invention.

### IV. Methods of Use

In particular embodiments, methods of the disclosure include the use of an effective amount of CAR-expressing fibroblasts to an individual in need thereof. In at least some cases, the individual has been diagnosed definitively for the medical condition or disease or the individual may be at risk for developing medical condition or disease because of personal and/or family history, being a smoker, being overweight or obese, having one or more genetic markers that predisposes the individual to the medical condition or disease, and so forth. The CAR-expressing fibroblasts may be provided to the individual at the first indication of the medical condition or disease, although in other cases they are provided upon a second or subsequent indication of the medical condition or disease. The individual may be administered the CAR-expressing fibroblasts as a first therapy for the medical condition or disease or after another therapy has already been administered to the individual.

The CARs of the disclosure may be used to overcome therapeutic failures arising from antigen loss escape variants, to reduce resistance to existing therapies and/or to treat diseases associated with the antigens targeted by the CARs.

Accordingly, the disclosure also provides methods for treating a disease associated with the antigen targeted by the CAR of the disclosure in a subject in need thereof. The method comprises providing a composition comprising the CAR-expressing fibroblasts of the disclosure and administering an effective amount of the composition so as to treat the disease associated with the antigen in the subject.

The disclosure also provides methods for overcoming therapeutic failures arising from antigen loss escape variants in disease states (e.g., B-cell diseases) in subjects in need thereof. The method comprises providing a composition comprising the CAR-expressing fibroblasts of the disclosure and administering an effective amount of the composition so as to treat the disease associated with the antigen in the subject.

In specific embodiments, the CAR-expressing fibroblast cells are administered to an individual in combination with one or more other therapies, and the other therapies may or may not be administered to the individual at the same time.

In some embodiments, the composition comprises a polynucleotide encoding the CAR, a protein comprising the CAR or genetically modified fibroblast cells comprising the CAR. The compositions of the disclosure may be administered alone or in conjunction with existing therapies. If other therapies are used in conjunction, the compositions of the disclosure may be administered concurrently or sequentially with the other the existing therapies.

In specific embodiments, the CAR-expressing fibroblast cells are administered once to the individual or multiple times to the individual. When the CAR-expressing fibroblast cells are administered multiple times, it may be with any suitable duration between administrations, such as within 1, 2, 3, 4, 5, 6, 7, 8, 9, 10, 11, 12, 13, 14, 15, 16, 17, 18, 19, 20, 21, 22, 23, or 24 hours of each other, or within 1, 2, 3, 4, 5, 6, or 7 days of each other, or within 1, 2, 3, or 4 weeks of each other, or within 1, 2, 3, 4, 5, 6, 7, 8, 9, 10, 11, or 12 months of each other, or within 1, 2, 3, 4, 5, 6, 7, 8, 9, or 10 or more years of each other.

“Cancer” and “cancerous” refer to or describe the physiological condition in mammals that is typically characterized by unregulated cell growth. Examples of cancer include, but are not limited to B-cell lymphomas (Hodgkin's lymphomas and/or non-Hodgkins lymphomas), brain tumor, breast cancer, colon cancer, lung cancer, hepatocellular cancer, gastric cancer, pancreatic cancer, cervical cancer, ovarian cancer, liver cancer, bladder cancer, cancer of the urinary tract, thyroid cancer, renal cancer, carcinoma, melanoma, head and neck cancer, brain cancer, stomach cancer, esophageal cancer, endometrial cancer, testicular cancer, uterine cancer, gall bladder cancer, thyroid cancer, and prostate cancer, including but not limited to androgen-dependent prostate cancer and androgen-independent prostate cancer. In cases wherein the individual has cancer, the CAR-expressing fibroblast cells may be administered in combination with chemotherapy, radiation, surgery, hormone therapy, proton therapy, immunotherapy, or a combination thereof.

The dosage of the CAR-expressing fibroblasts varies within wide limits and may be adjusted to the individual requirements in each particular case. The number of cells used depends on the age, weight, sex, and condition of the recipient, the number and/or frequency of administrations, the disease or disorder being treated, and the extent or severity thereof, and other variables known to those of skill in the art.

In a non-limiting embodiment, the CAR-expressing fibroblasts may be administered in combination with one or more other anti-cancer therapies. The therapy may include chemotherapy drug(s), radiation, surgery, hormone therapy, proton therapy, immune therapy, or a combination thereof, for example. Examples of drug(s) include alkylating agents such as ifosfamide, nimustine hydrochloride, cyclophosphamide, dacarbazine, melphalan, and ranimustine, antimetabolites such as gemcitabine hydrochloride, enocitabine, cytarabine ocfosfate, a cytarabine formulation, tegafur/uracil, a tegafur/gimeracil/oteracil potassium mixture, doxifluridine, hydroxycarbamide, fluorouracil, methotrexate, and mercaptopurine, antitumor antibiotics such as idarubicin hydrochloride, epirubicin hydrochloride, daunorubicin hydrochloride, daunorubicin citrate, doxorubicin hydrochloride, pirarubicin hydrochloride, bleomycin hydrochloride, peplomycin sulfate, mitoxantrone hydrochloride, and mitomycin C, alkaloids such as etoposide, irinotecan hydrochloride, vinorelbine tartrate, docetaxel hydrate, paclitaxel, vincristine sulfate, vindesine sulfate, and vinblastine sulfate, hormone therapy agents such as anastrozole, tamoxifen citrate, toremifene citrate, bicalutamide, flutamide, and estramustine phosphate, platinum complexes such as carboplatin, cisplatin, and nedaplatin, angiogenesis inhibitors such as thalidomide, neovastat, and bevacizumab, L-asparaginase, etc., drugs inhibiting the activity or production of the above bioactive substances, such as, for example, antibodies and antibody fragments that neutralize the above bioactive substances, and substances that suppress expression of the above bioactive substances, such as an siRNA, a ribozyme, an antisense nucleic acid (including RNA, DNA, PNA, and a composite thereof), substances that have a dominant negative effect such as a dominant negative mutant, vectors expressing same, cell activity inhibitors such as a sodium channel inhibitor, cell-growth inhibitors, and apoptosis inducers such as compound 861 and gliotoxin.

In one embodiment of the disclosure, fibroblasts are administered to a subject by any suitable route, including by injection (such as intramuscular injection), including in hypoxic areas. Suitable routes include intravenous, subcutaneous, intrathecal, oral, intrarectal, intrathecal, intra-omentral, intraventricular, intrahepatic, and intrarenal.

In particular embodiments, a suicide gene is utilized in conjunction with the fibroblast therapy to control its use and allow for termination of the cell therapy at a desired event and/or time. The suicide gene is employed in transduced fibroblast cells for the purpose of eliciting death for the transduced cells when needed. The cells of the present disclosure that have been modified to harbor a vector may comprise one or more suicide genes on the vector. In some embodiments, the term “suicide gene” as used herein is defined as a gene which, upon administration of a prodrug or other agent, effects transition of a gene product to a compound which kills its host cell. In other embodiments, a suicide gene encodes a gene product that is, when desired, targeted by an agent (such as an antibody) that targets the suicide gene product.

Examples of suicide gene/prodrug combinations which may be used are Herpes Simplex Virus-thymidine kinase (HSV-tk) and ganciclovir, acyclovir, or FIAU; oxidoreductase and cycloheximide; cytosine deaminase and 5-fluorocytosine; thymidine kinase thymidilate kinase (Tdk::Tmk) and AZT; and deoxycytidine kinase and cytosine arabinoside. The E. coli purine nucleoside phosphorylase, a so-called suicide gene that converts the prodrug 6-methylpurine deoxyriboside to toxic purine 6-methylpurine, may be used. Other examples of suicide genes used with prodrug therapy are the E. coli cytosine deaminase gene and the HSV thymidine kinase gene.

Exemplary suicide genes also include CD20, CD52, EGFRv3, or inducible caspase 9. In one embodiment, a truncated version of EGFR variant III (EGFRv3) may be used as a suicide antigen that can be ablated by Cetuximab. Further suicide genes known in the art that may be used in the present disclosure include Purine nucleoside phosphorylase (PNP), Cytochrome p450 enzymes (CYP), Carboxypeptidases (CP), Carboxylesterase (CE), Nitroreductase (NTR), Guanine Ribosyltransferase (XGRTP), Glycosidase enzymes, Methionine-α,γ-lyase (MET), and Thymidine phosphorylase (TP).

### V. Fibroblast Augmentation of Immunotherapy Including CAR-T Cell Through Suppression of Chronic Inflammation and Neutrophil Hyperactivity

Autologous cell therapies face substantial technical and logistic hurdles to practical application, their generation requires expensive dedicated facilities and expert personnel, they must be generated in a short time following a patient's diagnosis, and in many cases, pretreatment of the patient has resulted in degraded immune function, such that the patient's lymphocytes may be poorly functional and present in very low numbers. Because of these hurdles, each patient's autologous cell preparation is effectively a new product, resulting in substantial variations in efficacy and safety. Ideally, one would use a standardized therapy in which allogeneic therapeutic cells could be pre-manufactured, characterized in detail, and available for immediate administration to patients. By allogeneic it is meant that the cells are obtained from individuals belonging to the same species but are genetically dissimilar. However, the use of allogeneic cells presently has many drawbacks. In immune-competent hosts allogeneic cells are rapidly rejected, a process termed host versus graft rejection (HvG), and this substantially limits the efficacy of the transferred cells. Currently there is a need to improve efficacy of CAR-expressing cells of any kind, including CAR-expressing T cells, CAR-expressing NK cells, and CAR-expressing NKT cells.

In particular embodiments, there are means of suppressing cancer-associated inflammation that in its chronic form results in neutrophil hyperactivation as well as reduction of immune activities through oxidative stress-induced cleavage of the T cell receptor CD3 zeta chain. In some embodiments the disclosure, there is augmentation of CAR T cell activity through suppression of inflammation through administration of fibroblast cell populations possessing anti-inflammatory activity. Thus, in this particular embodiment, fibroblast cells are not CAR-expressing themselves, as other embodiments herein, but instead enhance methods of utilizing other non-fibroblast types of CAR-expressing cells.

The disclosure provides the overcoming of cancer-associated immune suppression by utilization of fibroblasts as anti-inflammatory cells. The disclosure provides means of paradoxically using fibroblasts to enhance immune activation. It is known that fibroblasts may act as immune suppressive cells. This would be counterintuitive for use in cancer, in which immune system upregulation is desired. However the inventors discovered that by suppressing neutrophil activity, in part through the administration of fibroblasts, the reduction in neutrophil activation allows for T cell and NK cell function in cancer patients to be upregulated. The disclosure recognizes that activated neutrophils, as well as other cells associated with inflammatory situations, result in producing a low level chronic oxidative stress that results in the inhibition of immunity. Thus, in specific embodiments, the administration of fibroblasts reduces neutrophil activation that enhances use of T cell and NK cell function in an individual in need thereof.

Embodiments of the disclosure include methods of augmenting activity of a chimeric antigen receptor (CAR)-specific cell, such as a CAR-specific T cell, NK cell and/or NKT cell. In specific embodiments, an effective amount of fibroblasts are provided to an individual that will receive, is receiving, or has received an effective amount of CAR-expressing cell therapy.

Embodiments of the disclosure include methods of augmenting activity of a chimeric antigen receptor (CAR) T cell by reversing cancer-associated immune suppression. In specific embodiments, the method comprises the steps of: a) selecting a cancer patient possessing one or more defects in the immune system; b) assessing the degree of chronic inflammation; and c) administering to said patient a concentration of fibroblasts capable of reducing chronic inflammation.

The CAR-expressing cell may be specific for a solid tumor and/or may be generated in a manner to recognize one or more tumor antigens. The tumor antigen(s) may be present on the surface of the tumor cells and do not require major histocompatibility type 1 molecules or major histocompatibility type 2 molecules for presentation. The tumor antigen(s) may be present on the surface of the tumor cells and require major histocompatibility type 1 molecules or major histocompatibility type 2 molecules for presentation. In specific embodiments, the tumor antigens are peptides or proteins selected from the group consisting of: a) Fos-related antigen 1; b) LCK; c) FAP; d) VEGFR2; e) NA17; f) PDGFR-beta; g) PAP; h) MAD-CT-2; i) Tie-2; j) PSA; k) protamine 2; l) legumain; m) endosialin; n) prostate stem cell antigen; o)carbonic anhydrase IX; p) STn; q) Page4; r) proteinase 3; s) GM3 ganglioside; t) tyrosinase; u) MART1; v) gp100; w) SART3; x) RGS5; y) SSX2; z) Globoll; aa) Tn; ab) CEA; ac) hCG; ad) PRAME; ae) XAGE-1; af) AKAP-4; ag) TRP-2; ah) B7H3; ai) sperm fibrous sheath protein; aj) CYP1B1; ak) HMWMAA; al) sLe(a); am) MAGE A1; an) GD2; ao) PSMA; ap) mesothelin; aq) fucosyl GM1; ar) GD3; as) sperm protein 17; at) NY-ESO-1; au) PAX5; av) AFP; aw) polysialic acid; ax) EpCAM; ay) MAGE-A3; az) mutant p53; ba) ras; bb) mutant ras; bc) NY-BR1; bd) PAX3; be) HER2/neu; bf) OY-TES1; bg) HPV E6 E7; bh) PLAC1; bi) hTERT; bj) BORIS; bk) ML-IAP; bl) idiotype of b cell lymphoma or multiple myeloma; bm) EphA2; bn) EGFRvIII; bo) cyclin B1; bp) RhoC; bq) androgen receptor; br) surviving; bs) MYCN; bt) wildtype p53; bu) LMP2; by) ETV6-AML; bw) MUC1; bx) BCR-ABL; by) ALK; bz) WT1; ca) ERG (TMPRSS2 ETS fusion gene); cb) sarcoma translocation breakpoint; cc) STEAP; cd) OFA/iLRP; ce) Chondroitin sulfate proteoglycan 4 (CSPG4); cd) and a combination thereof.

In particular embodiments, the CAR-expressing cells are modified to secrete one or more immunogenic cytokines, including immunogenic cytokines that are capable of increasing immune response towards tumor being targeted by the CAR-expressing cell. In specific cases, the immunogenic cytokines increase expression of one or more costimulatory molecules on tumor cells. In specific embodiments, the immunogenic cytokines increase expression of costimulatory molecules on antigen presenting cells adjacent to the tumor cells. The immunogenic cytokines are selected from the group consisting of: a) interleukin-1; b) interleukin-2; c) interleukin-3; d) interleukin-7; e) interleukin-12; f) interleukin-15; g) interleukin-18; h) interleukin-21; i) interleukin-23; j) interleukin-27; k) interleukin-33; l) tumor necrosis factor alpha; m) interferon alpha; n) interferon beta; o) interferon gamma; p) HMGB-1; q) Fas ligand; and r) a combination thereof.

Cancer-associated immune suppression may be manifested by reduced ability of T cells to produce cytokines capable of inhibiting cancer including cytokines that inhibit production of new blood vessels, such as those selected from the group consisting of: a) lymphotoxin; b) endostatin; c) interferon gamma; d) interferon gamma inducible protein-10; e) interleukin-18; and f) a combination thereof. In specific aspects, the cytokines capable of inhibiting cancer are cytokines which directly suppress proliferation and/or viability of cancer cells and may be selected from the group consisting of: a) granzyme; b) Fas ligand; c) tumor necrosis factor alpha; d) tumor necrosis factor beta; e) interleukin-10; f) angiostatin; and g) a combination thereof. In specific aspects, cytokines capable of inhibiting cancer are cytokines capable of activating T cells; capable of activating NK cells; capable of inducing M1 polarization in macrophages; capable of reducing M2 polarization in macrophages; capable of activating gamma delta T cells; capable of activating Natural kill T cells; and/or capable of inhibiting T regulatory cells (including at least T regulatory cells that express FoxP3; express surface TGF-beta; and/or that inhibit activation of T cells). In particular embodiments, cytokines capable of inhibiting cancer are cytokines capable of inhibiting B regulatory cells, including B regulatory cells that express the marker CD5. The B regulatory cells may be capable of producing interleukin-10.

In particular embodiments, the immune suppression associated with methods of the disclosure is associated with activated neutrophils that may be capable of producing hydrogen peroxide without further stimulation and/or that may be capable of producing superoxide radical without further stimulation. Activated neutrophils produce a greater amount of hydrogen peroxide when exposed to zymosan as compared to neutrophils isolated from a healthy, age-matched individual, in particular aspects. Activated neutrophils produce a greater amount of superoxide radical when exposed to zymosan as compared to neutrophils isolated from a healthy, age-matched individual, in particular aspects.

In certain embodiments, the immune suppression associated with methods of the disclosure is quantified by reduced ability of T cells to proliferate in response to a signal crosslinking CD3 and a secondary signal crosslinking a secondary signal molecule, wherein the secondary signal molecular may be CD28; IL-2 receptor; IL-7 receptor; IL-15 receptor; CD40 ligand; 4.1-BB; ICOS; or a combination thereof.

In specific embodiments, immune suppression is quantified by reduced ability of T cells to proliferate in response stimulation with phytohemagluttanin; immune suppression may be quantified by reduced ability of T cells to proliferated in response stimulation beads containing CD3 and CD28 crosslinking antibodies; and/or immune suppression may be quantified by reduced ability of T cells to proliferated in response stimulation by allogeneic antigen presenting cells. The allogeneic antigen presenting cells may be tumor cells stimulated with interferon gamma; B cells; macrophages; and/or dendritic cells, in specific embodiments.

In certain embodiments, the immune suppression comprises reduction of activity of natural killer cells, and the reduction of activity of the natural killer cells may be quantified by the ability of the natural killer cells to induce death of cells expressing low or absent levels of major histocompatibility molecules. In specific cases, cells expressing low or absent levels of major histocompatibility molecules are neoplastically transformed cells, such as K-562 erythroleukemia cell lines.

In particular embodiments, the cancer associated immune suppression is associated with chronic inflammation that may be quantified by assessment of plasma levels of C-Reactive Protein; assessment of plasma levels of HMGB-1; assessment of plasma levels of interleukin-17; assessment of plasma levels of malondialdehyde; assessment of plasma levels of glutathione; assessment of plasma levels of oxidized LDL; assessment of plasma levels of superoxide dismutase; assessment of plasma levels of catalase; and/or assessment of plasma levels of adropin.

In specific cases, chronic inflammation is addressed by administration of one or more agents capable of reducing chronic inflammation before administration of fibroblasts, and the agent(s) may be selected from the group consisting of: NSAIDs, interleukin-1 antagonists, dihydroorotate synthase inhibitors, p38 MAP kinase inhibitors, TNF-.alpha. inhibitors, TNF-.alpha. sequestration agents, and methotrexate. More specifically, anti-inflammatory agents may comprise one or more of, e.g., anti-TNF-.alpha., lysophylline, alpha 1-antitrypsin (AAT), interleukin-10 (IL-10), pentoxyfilline, COX-2 inhibitors, 21-acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortamate, hydrocortisone, loteprednol etabonate, mazipredone, medrysone, meprednisone, methylprednisolone, mometasone furoate, paramethasone, prednicarbate, prednisolone, prednisolone 25-diethylamino-acetate, prednisolone sodium phosphate, prednisone, prednival, prednylidene, rimexolone, tixocortol, triamcinolone, triamcinolone acetonide, triamcinolone benetonide, triamcinolone hexacetonide, aminoarylcarboxylic acid derivatives (e.g., enfenamic acid, etofenamate, flufenamic acid, isonixin, meclofenamic acid, mefenamic acid, niflumic acid, talniflumate, terofenamate, tolfenamic acid), arylacetic acid derivatives (e.g., aceclofenac, acemetacin, alclofenac, amfenac, amtolmetin guacil, bromfenac, bufexamac, cinmetacin, clopirac, diclofenac sodium, etodolac, felbinac, fenclozic acid, fentiazac, glucametacin, ibufenac, indomethacin, isofezolac, isoxepac, lonazolac, metiazinic acid, mofezolac, oxametacine, pirazolac, proglumetacin, sulindac, tiaramide, tolmetin, tropesin, zomepirac), arylbutyric acid derivatives (e.g., bumadizon, butibufen, fenbufen, xenbucin), arylcarboxylic acids (e.g., clidanac, ketorolac, tinoridine), arylpropionic acid derivatives (eg., alminoprofen, benoxaprofen, bermoprofen, bucloxic acid, carprofen, fenoprofen, flunoxaprofen, flurbiprofen, ibuprofen, ibuproxam, indoprofen, ketoprofen, loxoprofen, naproxen, oxaprozin, piketoprolen, pirprofen, pranoprofen, protizinic acid, suprofen, tiaprofenic acid, ximoprofen, zaltoprofen), pyrazoles (e.g., difenamizole, epirizole), pyrazolones (e.g., apazone, benzpiperylon, feprazone, mofebutazone, morazone, oxyphenbutazone, phenylbutazone, pipebuzone, propyphenazone, ramifenazone, suxibuzone, thiazolinobutazone), salicylic acid derivatives (e.g., acetaminosalol, aspirin, benorylate, bromosaligenin, calcium acetylsalicylate, diflunisal, etersalate, fendosal, gentisic acid, glycol salicylate, imidazole salicylate, lysine acetylsalicylate, mesalamine, morpholine salicylate, 1-naphthyl salicylate, olsalazine, parsalmide, phenyl acetylsalicylate, phenyl salicylate, salacetamide, salicylamide o-acetic acid, salicylsulfuric acid, salsalate, sulfasalazine), thiazinecarboxamides (e.g., ampiroxicam, droxicam, isoxicam, lornoxicam, piroxicam, tenoxicam), epsilon.-acetamidocaproic acid, s-adenosylmethionine, 3-amino-4-hydroxybutyric .acid, amixetrine, bendazac, benzydamine, .alpha.-bisabolol, bucolome, difenpiramide, ditazol, emorfazone, fepradinol, guaiazulene, nabumetone, nimesulide, oxaceprol, paranyline, perisoxal, proquazone, superoxide dismutase, tenidap, zileuton, candelilla wax, alpha bisabolol, aloe vera, Manjistha, Guggal, kola extract, chamomile, sea whip extract, glycyrrhetic acid, glycyrrhizic acid, oil soluble licorice extract, monoammonium glycyrrhizinate, monopotassium glycyrrhizinate, dipotassium glycyrrhizinate, 1-beta-glycyrrhetic acid, stearyl glycyrrhetinate, and 3-stearyloxy-glycyrrhetinic acid. In specific embodiments, an immune stimulator capable of activating innate immunity is provided to the individual in order to concurrently reduce cancer immune suppression while activating the non-adaptive part of the immune system. In specific cases, the stimulator of immunity which is non-adaptive is a toll like receptor agonist, and the toll like receptor may be selected from the group of toll like receptors consisting of: TLR-1, TLR-2, TLR-3, TLR-4, TLR-5, TLR-6, TLR-7, TLR-8, TLR-9, and a combination thereof.

In specific embodiments, an activator of said toll like receptor agonist is selected from the group consisting of: Pam3CSK4, HKLM, Poly:IC, LPS, Buprenorphine, Carbamazepine, Fentanyl, Le-vorphanol, Methadone, Cacaine, Morphine, Oxcarbazepine, Oxycodone, Pethidine, Glucuronox-ylomannan from Cryptococcus, Morphine-3-glucuronide, lipoteichoic acid, β-defensin 2, small molecular weight hyaluronic acid, fibronectin EDA, snapin, tenascin C, flagellin, FSL-1, imiquimod, ssRNA40/LyoVec CpG oligonucleotide, ODN2006, and Agatolimod.

In specific embodiments, the toll like receptor agonist(s) is administered in a manner to induce systemic activation of antigen presenting cells that may be selected from the group consisting of: a) dendritic cells; b) monocytes; c) macrophages; d) B cells; and e) a combination thereof. The toll like receptor agonist may be administered in a manner to induce systemic activation of natural killer cells.

In specific embodiments, activators of innate immunity are administered in a manner to promote intratumoral shift of macrophage activity from M2 to M1. The M1 macrophages may be cytotoxic macrophages capable of phagocytosing tumor cells; capable of mediating antibody dependent cellular toxicity; and/or capable of producing higher amounts of nitric oxide upon their activation as compared to arginase. The M1 macrophages may inhibit angiogenesis in tumors. In specific embodiments, the M2 macrophages promote tumor angiogenesis. In specific aspects, the M2 macrophages possess higher arginase activity as compared to nitric oxide activity.

In specific embodiments, a stimulator of innate immunity induces an acute upregulation of genes selected from the group consisting of: IL-6, Myosin 1, IL-33, Hypoxia Inducible Factor-1, Guanylate Binding Protein Isoform I, Aminolevulinate delta synthase 2, AMP deaminase, IL-17, DNAJ-like 2 protein, Cathepsin L, Transcription factor-20, M31724, pyenylalkylamine binding protein; HEC, GA17, arylsulfatase D gene, arylaulfatase E gene, cyclin protein gene, pro-platelet basic protein gene, PDGFRA, human STS WI-12000, mannosidase, beta A, lysosomal MANBA gene, UBE2D3 gene, Human DNA for Ig gamma heavy-chain, STRL22, BHMT, Homo sapiens Down syndrome critical region, FI5613 containing ZNF gene family member, IL8, ELFR, Homo sapiens mRNA for dual specificity phosphatase MKP-5, Homo sapiens regulator of G protein signaling 10 mRNA complete, Homo sapiens Wnt-13 Mma, Homo sapiens N-terminal acetyltransferase complex ard1 subunit, ribosomal protein L15 mRNA, PCNA mRNA, ATRM gene exon 21, HR gene for hairless protein exon 2, N-terminal acetyltransferase complex and 1 subunit, HSM801431 Homo sapiens mRNA, CDNA DKFZp434N2072, RPL26, HR gene for hairless protein, regulator of G protein signaling, and a combination thereof.

In one embodiment, augmentation of expression of genes is observed in monocytes taken from said patient.

In particular embodiments, fibroblasts possessing anti-inflammatory properties are autologous to an individual in need of treatment. Fibroblasts possessing anti-inflammatory properties may be allogeneic to the individual in need of treatment. Anti-inflammatory properties of the fibroblast may include the ability to reduce production of TNF-alpha from antigen presenting cells, such as monocytes and/or endothelial cells. Anti-inflammatory properties of the fibroblasts include an ability to reduce levels of C reactive protein in circulation; ability to enhance levels of superoxide dismutase in circulation; ability to enhance levels of glutathione in circulation; ability to enhance levels of catalase in circulation; and/or ability to reduce levels of IL-17 in circulation.

In particular embodiments, fibroblasts are cultured and administered in a proliferating phase. The fibroblasts may be plastic adherent. The fibroblasts may be capable of producing one or more cytokines, such as are selected from the group consisting of: a) IL-10; b) TGF-beta; c) IL-4; d) IL-13; f) VEGF; and g) a combination thereof. The fibroblasts may be capable of reducing proliferation in a mixed lymphocyte reaction and/or capable of reducing cytokine production in a mixed lymphocyte reaction. A cytokine whose production may be suppressed by the fibroblasts may be selected from the group consisting of: a) interferon gamma; b) TNF-alpha; c) interleukin-33; d) interleukin-18; and 3) a combination thereof. The fibroblasts may be capable of enhance cytokine production in a mixed lymphocyte reaction, and the cytokine(s) whose level is enhanced may be anti-inflammatory. Anti-inflammatory cytokines may be selected from the group consisting of: a) IL-10; b) TGF-beta; c) IL-13; and d) a combination thereof.

Immune suppression may be quantified by cleavage of the T cell receptor zeta chain, and the cleavage of the zeta chain may be evoked by activation of caspase 3 in T cells.

The disclosure additionally provides pharmaceutical compositions comprising the fibroblast cells that are administered prior to, and/or concurrent with, and/or subsequent to, CAR-expressing cell administration. In one embodiment of the disclosure, the pharmaceutical composition comprises an effective amount of one or more fibroblast cells of the disclosure and one or more pharmaceutically acceptable carriers. In some embodiments, combinations of cells is utilized. Combinations envisioned include fibroblast cells administered with immune cells, in some embodiments, other adoptive immunotherapy approaches in addition to CAR-expressing cells are utilized in conjunction with fibroblast immunotherapy. Encompassed fibroblast cells of the disclosure and compositions comprising the cells can be conveniently provided in sterile liquid preparations, for example, typically isotonic aqueous solutions with cell suspensions, or optionally as emulsions, dispersions, or the like, which are typically buffered to a selected pH. The compositions can comprise carriers, for example, water, saline, phosphate buffered saline, and the like, suitable for the integrity and viability of the cells, and for administration of a cell composition.

In one embodiment, the invention encompasses determination of the state of immune suppression in individuals suffering from cancer, and this may be performed according to some of the means and methods provided in the literature and incorporated by reference herein. For example, in one embodiment assessment of neutrophil activation is performed in patients that are eligible for CAR-expressing cell therapy. In one embodiment, neutrophils in the form of granulocytes are purified from peripheral blood with dextran sedimentation. Peripheral blood mononuclear cells (PBMC) are collected by centrifugation in lymphocyte separation medium (ICN), and residual RBCs are lysed in a hypotonic saline solution. To obtain activated granulocytes, whole blood is incubated for 1 h with FMLP prior to density gradient centrifugation. Cells are stained with anti-CD15 antibody for flow cytometry or used for cytospins stained with Diff-Quick. T cells may be activated for 4 h with 20 μg/mL PMA and 1 μm ionomycin. Additionally, T cells may be activated with other means known in the art including culture with anti-CD3 anti-CD28 beads, or by exposure to a mitogen such as a lectin. Commonly used mitogens for activation of T cells including phytohemagglutinin, conconanvalin A, and pokeweed mitogen.

In another embodiment of the disclosure, the TCR-zeta chain cleavage is assessed as means of quantifying immune suppression. PBMC samples can analyzed for TCRζ expression and cytokine production by flow cytometry. For intracellular cytokine staining, 2 mm monensin is added to T cells for 4 h. Cells are fixed with 2% PFA³ and permeabilized with FACS buffer (PBS supplemented with 5% FBS and 0.1% sodium azide) containing 0.1% saponin. An anti-TCRζ chain antibody, TCR-ζ TIA-2 was used for indirect staining prior to a secondary goat antimouse R-phycoerythrin-conjugated antibody. Antibodies against the cytokines IFN-γ, TNF-α, IL-2, or IL-4 were obtained from PharMingen. Live cells were stained with antibodies against CD11b, CD14, and CD15 on granulocytes and monocytes and antibodies against CD3 and CD8 on T cells. Analysis was done on a FACS-Calibur. This is one representative assay, but other assays are known in the art for detection of cytokine production ability, as well as for cleavage of the TCR zeta chain.

In some embodiments of the disclosure, the assessment of immune suppressive activity of granulocytes such as neutrophils is assessed by performing granulocyte/T-cell co-culture assays, both cell types may be purified from the same blood sample. Granulocytes (4×10⁶) are seeded into the lower compartment of a six-well transwell system, and 2×10⁶ PBMCs are either placed into the upper compartment or directly mixed with the granulocytes in a final volume of 5 mL. The experiment, involving freezing, fixation, or addition of bovine catalase is performed with 7.5×10⁵ T cells and granulocytes in a 24-well plate (Costar). Granulocytes were either frozen overnight in FBS with 10% DMSO at −80° C. or fixed in 2% PFA/PBS for 20 min prior to the assay. For experiments involving H₂O₂, PBMCs were treated for 5 min, followed by an additional 5-min incubation with catalase at a final concentration of 1000 units/mL. Dose-response studies with H₂O₂ were done in the absence of fetal calf serum. These studies can be utilized to assess ability of the granulocytes to suppress T cell activity, to cleave zeta chain, and also to quantify how much of this is dependent on oxidative stress. These assays may also be utilized to detect the therapeutic efficacy of the fibroblast administration.

In some embodiments, fibroblast cells are isolated from a sample or biopsy of bodily tissue by digestion by enzymatic digestion, mechanical separation, filtration, centrifugation and combinations thereof. The number and quality of the isolated fibroblast cells can vary depending, e.g., on the quality of the tissue used, the compositions of perfusion buffer solutions, and the type and concentration of enzyme. Frequently used enzymes include, but are not limited to, collagenase, pronase, trypsin, dispase, hyaluronidase, thermolysin and pancreatin, and combinations thereof. Collagenase is most commonly used, often prepared from bacteria (e.g. from Clostridium histolyticum), and may often consist of a poorly purified blend of enzymes, which may have inconsistent enzymatic action. Some of the enzymes exhibit protease activity, which may cause unwanted reactions affecting the quality and quantity of viable/healthy fibroblast cells. It is understood by those of skill in the art to use enzymes of sufficient purity and quality to obtain viable fibroblast cell populations.

The methods of the studies comprise culturing fibroblast cells obtained from human tissue samples. In one embodiment, the populations of fibroblast cells are plated onto a substrate. In the present disclosure, fibroblasts are plated onto a substrate that allows for adherence of cells thereto. This may be carried out, e.g., by plating the cells in a culture plate which displays one or more substrate surfaces compatible with cell adhesion. When the one or more substrate surfaces contact the suspension of cells (e.g., suspension in a medium) introduced into the culture system, cell adhesion between the cells and the substrate surfaces may ensue. Accordingly, the term “plating onto a substrate which allows adherence of cells thereto” refers to introducing cells into a culture system that features at least one substrate surface that is generally compatible with adherence of cells thereto, such that the plated cells can contact the said substrate surface. General principles of maintaining adherent cell cultures are well-known in the art. As appreciated by those skilled in the art, the fibroblast cells may be counted in order to facilitate subsequent plating of the cells at a desired density. Where, as in the present disclosure, the cells after plating may primarily adhere to a substrate surface present in the culture system (e.g., in a culture vessel), the plating density may be expressed as number of cells plated per mm² or cm² of the said substrate surface. In practicing the invention, after plating of the fibroblasts, the cell suspension is left in contact with the adherent surface to allow for adherence of cells from the cell population to the said substrate. In contacting fibroblasts to the adherent substrate, the cells may be advantageously suspended in an environment comprising at least a medium, in the methods of the invention typically a liquid medium, which supports the survival and/or growth of the cells. The medium may be added to the system before, together with or after the introduction of the cells thereto. The medium may be fresh, i.e., not previously used for culturing of cells, or may comprise at least a portion which has been conditioned by prior culturing of cells therein, e.g., culturing of the cells which are being plated or antecedents thereof, or culturing of cells more distantly related to or unrelated to the cells being plated.

Cells of the disclosure include fibroblasts from various tissues, selected for specific properties associated with regenerative activity. Tissues useful for the practice of the invention are generally tissues associated with regenerative activity. Te tissues include placenta, endometrial cells, Wharton's jelly, bone marrow, and adipose tissue. In a particular embodiment, cells are selected for expression of the markers CD117, CD105, and expression of the rhodamine 123 efflux activity. In some embodiments of the disclosure, fibroblasts are selected for expression of markers selected from the group consisting of additional markers Oct-4, CD-34, KLF-4, Nanog, Sox-2, Rex-1, GDF-3, Stella, and that possesses enhanced expression of GDF-11. Selection of fibroblasts for expression of the markers may be performed by initial expression of proteins found on the membrane of the cells, which result in possessing other markers mentioned.

The medium may be a suitable culture medium as described elsewhere in this specification. In particular, the composition of the medium may have the same features, may be the same or substantially the same as the composition of medium used in the ensuing steps of culturing the attached cells. Otherwise, the medium may be different. The cells from the fibroblast cell population or from tissue explants of the present disclosure, which have adhered to the said substrate, preferably in the said environment, are subsequently cultured for at least 7 days, for at least 8 days, or for at least 9 days, for at least 10 days, at least 11, or at least 12 days, at least 13 days or at least 14 days, for at least 15 days, for at least 16 days or for at least 17 days, or even for at least 18 days, for at least 19 days or at least 21 days or more. The term “culturing” is common in the art and broadly refers to maintenance and/or growth of cells and/or progeny thereof.

In some embodiments, the fibroblast cells may be cultured for at least between about 10 days and about 40 days, for at least between about 15 days and about 35 days, for at least between about 15 days and 21 days, such as for at least about 15, 16, 17, 18, 19 or 21 days. In some embodiments, the stem cells of the invention may be cultured for no longer than 60 days, or no longer than 50 days, or no longer than 45 days. The tissue explants and fibroblasts are cultured in the presence of a liquid culture medium. Typically, the medium will comprise a basal medium formulation as known in the art. Many basal media formulations can be used to culture fibroblasts herein, including but not limited to Eagle's Minimum Essential Medium (MEM), Dulbecco's Modified Eagle's Medium (DMEM), alpha modified Minimum Essential Medium (alpha-MEM), Basal Medium Essential (BME), Iscove's Modified Dulbecco's Medium (IMDM), BGJb medium, F-12 Nutrient Mixture (Ham), Liebovitz L-15, DMEM/F-12, Essential Modified Eagle's Medium (EMEM), RPMI-1640, and modifications and/or combinations thereof. Compositions of the above basal media are generally known in the art and it is within the skill of one in the art to modify or modulate concentrations of media and/or media supplements as necessary for the fibroblasts cultured. In some embodiments, a culture medium formulation may be explants medium (CEM) which is composed of IMDM supplemented with 10% fetal bovine serum (FBS, Lonza), 100 U/mL penicillin G, 100 .mu.g/mL streptomycin and 2 mmol/L L-glutamine (Sigma-Aldrich). Other embodiments may employ further basal media formulations, such as chosen from the ones above.

For use in the fibroblast culture, media can be supplied with one or more further components. For example, additional supplements can be used to supply the cells with the necessary trace elements and substances for optimal growth and expansion. Such supplements include insulin, transferrin, selenium salts, and combinations thereof. These components can be included in a salt solution such as, but not limited to, Hanks' Balanced Salt Solution (HBSS), Earle's Salt Solution. Further antioxidant supplements may be added, e.g., beta-mercaptoethanol. While many media already contain amino acids, some amino acids may be supplemented later, e.g., L-glutamine, which is known to be less stable when in solution. A medium may be further supplied with antibiotic and/or antimycotic compounds, such as, typically, mixtures of penicillin and streptomycin, and/or other compounds, exemplified but not limited to, amphotericin, ampicillin, gentamicin, bleomycin, hygromycin, kanamycin, mitomycin, mycophenolic acid, nalidixic acid, neomycin, nystatin, paromomycin, polymyxin, puromycin, rifampicin, spectinomycin, tetracycline, tylosin, and zeocin. Also contemplated is supplementation of cell culture medium with mammalian plasma or sera. Plasma or sera often contain cellular factors and components that are necessary for viability and expansion. The use of suitable serum replacements is also contemplated (e.g., FBS). As described, culturing tissue explants and fibroblast cells for time durations as defined above, and using media compositions as described above, a progenitor or stem cell of the invention may emerge and proliferate. In some embodiments, fibroblast cells of the present invention are identified and characterized by their expression of specific marker proteins, such as cell-surface markers. Detection and isolation of these cells can be achieved, e.g., through flow cytometry, ELISA, and/or magnetic beads. Reverse-transcription polymerase chain reaction (RT-PCR) can also be used to monitor changes in gene expression in response to differentiation. Methods for characterizing fibroblasts of the present invention are provided herein. In certain embodiments, the marker proteins used to identify and characterize the fibroblasts are selected from the list consisting of c-Kit, Nanog, Sox2, Hey1, SMA, Vimentin, Cyclin D2, Snail, E-cadherin, Nkx2.5, GATA4, CD105, CD90, CD29, CD73, Wt1, CD34, and CD45.

Embodiments of the present disclosure concern administering fibroblasts, including CAR-expressing fibroblasts, to an individual. In some embodiments, fibroblasts are administered before, with, or during the administration of CAR-expressing cells, such as CAR-expressing T cells, NKT cells, NK cells, and/or fibroblasts. The fibroblasts may augment the activity of the CAR-expressing cells. The fibroblasts may increase the CAR-expressing cells ability to induce an immune response, including an immune response against cancer cells expressing antigens targeted by the CAR-expressing cell. The fibroblasts administered before, with, or during the administration of CAR-expressing cells may be modified or may not be modified. Modified fibroblasts, including any modified fibroblast encompassed herein, may be cultured to modify the fibroblast. The modified fibroblast may have activity to induce an immune response that is higher than that in an unmodified fibroblast. In some embodiments, the fibroblasts are activated, dedifferentiated, reprogrammed, or a combination thereof before being administered to an individual.

### VI. Pharmaceutical Compositions

In various embodiments, the present disclosure provides pharmaceutical compositions comprising a pharmaceutically acceptable excipient and a therapeutically effective amount of the CAR-expressing fibroblasts of the disclosure. The CAR of the disclosure in the composition may be any one or more of a polynucleotide encoding the CAR, a protein comprising the CAR or genetically modified fibroblast cells comprising the CAR. The composition may further comprise polynucleotides encoding one or more other proteins co-expressed with the CAR or genetically modified fibroblast cells that express the CAR and one or more other proteins. “Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients may be solid, liquid, semisolid, or, in the case of an aerosol composition, gaseous.

In various embodiments, the present disclosure provides pharmaceutical compositions comprising a pharmaceutically acceptable excipient and a therapeutically effective amount of fibroblasts and CAR-expressing cells of the disclosure. The CAR of the disclosure in the composition may be any one or more of a polynucleotide encoding the CAR, a protein comprising the CAR or genetically modified cells comprising the CAR. The composition may further comprise polynucleotides encoding one or more other proteins co-expressed with the CAR or genetically modified cells that express the CAR and one or more other proteins.

In various embodiments, the pharmaceutical compositions according to the disclosure may be formulated for delivery via any route of administration. “Route of administration” may refer to any systemic or local administration pathway known in the art, including but not limited to aerosol, nasal, oral, intravenous, intramuscular, intraperitoneal, inhalation, transmucosal, transdermal, parenteral, implantable pump, continuous infusion, topical application, capsules and/or injections.

The pharmaceutical compositions according to the disclosure can also comprise any pharmaceutically acceptable carrier. “Pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

The pharmaceutical compositions according to the disclosure can also be encapsulated, tableted or prepared in an emulsion or syrup for oral administration. They can be encased in a syringe for injection. Pharmaceutically acceptable solid or liquid carriers may be added to enhance or stabilize the composition, or to facilitate preparation of the composition. Liquid carriers include syrup, peanut oil, olive oil, glycerin, saline, alcohols and water. Solid carriers include starch, lactose, calcium sulfate, dihydrate, terra alba, magnesium stearate or stearic acid, talc, pectin, acacia, agar or gelatin. The carrier may also include a sustained release material such as glyceryl monostearate or glyceryl distearate, alone or with a wax.

The pharmaceutical preparations may be made following the conventional techniques of pharmacy involving milling, mixing, granulation, and compressing, when necessary, for tablet forms; or milling, mixing and filling for hard gelatin capsule forms. When a liquid carrier is used, the preparation will be in the form of syrup, elixir, emulsion or an aqueous or non-aqueous suspension. Such a liquid formulation may be administered directly or filled into a soft gelatin capsule.

The pharmaceutical compositions according to the disclosure may be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20th edition, Williams & Wilkins Pa., USA) (2000).

### VII. Kits

Any of the cellular and/or non-cellular compositions described herein or similar thereto may be comprised in a kit. In a non-limiting example, one or more reagents for use in methods for preparing cellular therapy may be comprised in a kit. Such reagents may include fibroblast cells, vectors encoding a CAR or nucleic acid or polypeptide components that allow production of a CAR or a region thereof, one or more growth factors, vector(s) one or more costimulatory factors, media, enzymes, buffers, nucleotides, salts, primers, and so forth. The kit components are provided in suitable container means.

Some components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present disclosure also will typically include a means for containing the components in close confinement for commercial sale. Such containers may include injection or blow molded plastic containers into which the desired vials are retained.

When the components of the kit are provided in one and/or more liquid solutions, the liquid solution is an aqueous solution, with a sterile aqueous solution being particularly useful. In some cases, the container means may itself be a syringe, pipette, and/or other such like apparatus, or may be a substrate with multiple compartments for a desired reaction.

Some components of the kit may be provided as dried powder(s). When reagents and/or components are provided as a dry powder, the powder can be reconstituted by the addition of a suitable solvent. It is envisioned that the solvent may also be provided in another container means. The kits may also comprise a second container means for containing a sterile acceptable buffer and/or other diluent.

In specific embodiments, reagents and materials include primers for amplifying desired sequences, nucleotides, suitable buffers or buffer reagents, salt, and so forth, and in some cases the reagents include apparatus or reagents for isolation of a particular desired cell(s).

In particular embodiments, there are one or more apparatuses in the kit suitable for extracting one or more samples from an individual. The apparatus may be a syringe, fine needles, scalpel, and so forth.

### EXAMPLES

The following example is included to demonstrate particular embodiments of the disclosure. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Suppression of Leukemia Cell Proliferation by Car-Fibroblasts

Fibroblasts isolated from foreskin and growth under Opti-MEM media with 10% fetal calf serum were transfected with a chimeric antigen receptor construct targeting CD19, as an example of a target for the CAR. The CD19 targeting construct was developed as described by Brentjens et al. (Clin Cancer Res; 2007 Sep. 15; 13(18 Pt 1):5426-35) (as one example). The construct was transfected into fibroblasts.

VSV-G pseudotyped retroviral supernatants derived from transduced gpg29 fibroblasts were used to construct stable PG-13 gibbon ape leukemia virus (GaLV) envelope-pseudotyped retroviral-producing cell lines using polybrene (Sigma). The fusion receptors comprise the scFv derived from 19z1 as an example. All receptor constructs were generated using overlapping PCR. The resulting cassettes were designed to facilitate the exchange of the transmembrane and signaling domains of the 19z1 construct by NotI/BamHI restriction sites encoded in flanking primers.

Transfected fibroblasts were cultured with Raji cells that are known to express CD19. Interestingly, a dose-dependent suppression of proliferation was observed in co-culture of transfected fibroblasts but not control fibroblasts. Proliferation was assessed by thymidine incorporated 48 hours after co-culture. Concentration of Raji Cells was 40,000 cells per well

### REFERENCES

All patents and publications mentioned in the specification are indicative of the level of those skilled in the art to which the invention pertains. All patents and publications are herein incorporated by reference to the same extent as if each individual publication was specifically and individually indicated to be incorporated by reference.
1. Bland, P. W., The local immune response to large bowel tumors. Acta Chir Scand Suppl, 1985. 525: p. 70-92.
2. Wolf, G. T., et al., Lymphocyte subpopulations infiltrating squamous carcinomas of the head and neck: correlations with extent of tumor and prognosis. Otolaryngol Head Neck Surg, 1986. 95(2): p. 142-52.
3. Murali, P. S., et al., Interleukin-2 mediated regulation of mitogen-activated T cell reactivity from different lymphoid sources in patients with squamous cell carcinoma of the oral cavity. J Oral Pathol Med, 1989. 18(6): p. 327-32.
4. Tsujihashi, H., et al., Immunocompetence of tissue infiltrating lymphocytes in bladder tumors. J Urol, 1988. 140(4): p. 890-4.
5. Tsujihashi, H., et al., Immunohistochemical detection of tissue-infiltrating lymphocytes in bladder tumors. Urol Int, 1989. 44(1): p. 5-9.
6. Tsujihashi, H., et al., Immunoresponse of tissue infiltrating lymphocytes in bladder tumors. J Urol, 1989. 141(6): p. 1467-70.
7. Kuppner, M. C., M. F. Hamou, and N. de Tribolet, Immunohistological and functional analyses of lymphoid infiltrates in human glioblastomas. Cancer Res, 1988. 48(23): p. 6926-32.
8. Ebato, M., et al., Skewed distribution of TCR V alpha 7-bearing T cells within tumor-infiltrating lymphocytes of HLA-A24(9)-positive patients with malignant glioma. Immunol Lett, 1993. 39(1): p. 53-64.
9. Topalian, S. L., D. Solomon, and S. A. Rosenberg, Tumor-specific cytolysis by lymphocytes infiltrating human melanomas. J Immunol, 1989. 142(10): p. 3714-25.
10. Albertini, M. R., et al., Analysis of T cell receptor beta and gamma genes from peripheral blood, regional lymph node and tumor-infiltrating lymphocyte clones from melanoma patients. Cancer Immunol Immunother, 1991. 32(5): p. 325-30.
11. Pisani, R. J., et al., Lymphokine-activated killer (LAK) cell activity in tumor-infiltrating lymphocytes from non-small cell lung cancer. Am J Clin Pathol, 1989. 92(4): p. 435-46.
12. Yoshino, I., et al., Oligoclonal T lymphocytes infiltrating human lung cancer tissues. Int J Cancer, 1991. 47(5): p. 654-8.
13. Minamoto, T., et al., Medullary carcinoma with lymphocytic infiltration of the stomach. Clinicopathologic study of 27 cases and immunohistochemical analysis of the subpopulations of infiltrating lymphocytes in the tumor. Cancer, 1990. 66(5): p. 945-52.
14. Ma, D. and M. J. Gu, Immune effect of tumor-infiltrating lymphocytes and its relation to the survival rate of patients with ovarian malignancies. J Tongji Med Univ, 1991. 11(4): p. 235-9.
15. Peoples, G. E., et al., T-cell recognition of ovarian cancer. Surgery, 1993. 114(2): p. 227-34.
16. Kooi, S., et al., Cytokine production by T-cell lines derived from tumor-infiltrating lymphocytes from patients with ovarian carcinoma: tumor-specific immune responses and inhibition of antigen-independent cytokine production by ovarian tumor cells. Lymphokine Cytokine Res, 1993. 12(6): p. 429-37.
17. Di Giorgio, A., et al., The influence of tumor lymphocytic infiltration on long term survival of surgically treated colorectal cancer patients. Int Surg, 1992. 77(4): p. 256-60.
18. Horn, S. S., S. A. Rosenberg, and S. L. Topalian, Specific immune recognition of autologous tumor by lymphocytes infiltrating colon carcinomas: analysis by cytokine secretion. Cancer Immunol Immunother, 1993. 36(1): p. 1-8.
19. Oratz, R., et al., Induction of tumor-infiltrating lymphocytes in human malignant melanoma metastases by immunization to melanoma antigen vaccine. J Biol Response Mod, 1989. 8(4): p. 355-8.
20. Steerenberg, P. A., et al., Tumor infiltrating leukocytes (tils) during progressive tumor growth and BCG-mediated tumor regression. Virchows Arch B Cell Pathol Incl Mol Pathol, 1990. 59(4): p. 185-94.
21. Sawamura, Y. and N. de Tribolet, Immunobiology of brain tumors. Adv Tech Stand Neurosurg, 1990. 17: p. 3-64.
22. Topalian, S. L. and S. A. Rosenberg, Tumor-infiltrating lymphocytes: evidence for specific immune reactions against growing cancers in mice and humans. Important Adv Oncol, 1990: p. 19-41.
23. Knisely, T. L. and J. Y. Niederkorn, Immunologic evaluation of spontaneous regression of an intraocular murine melanoma. Invest Ophthalmol Vis Sci, 1990. 31(2): p. 247-57.
24. Kitai, H., et al., Spontaneous regression of small cell lung cancer combined with cancer associated retinopathy. Lung Cancer, 2015. 87(1): p. 73-6.
25. Bulkley, G. B., et al., Long-term spontaneous regression of malignant melanoma with visceral metastases. Report of a case with immunologic profile. Cancer, 1975. 36(2): p. 485-94.
26. Cole, W. H., Relationship of causative factors in spontaneous regression of cancer to immunologic factors possibly effective in cancer. J Surg Oncol, 1976. 8(5): p. 391-411.
27. Bodurtha, A. J., et al., A clinical histologic, and immunologic study of a case of metastatic malignant melanoma undergoing spontaneous remission. Cancer, 1976. 37(2): p. 735-42.
28. Hellstrom, K. E. and I. Hellstrom, Spontaneous tumor regression: possible relationship to in vitro parameters of tumor immunity. Natl Cancer Inst Monogr, 1976. 44: p. 131-4.
29. Firminger, H. I., A pathologist looks at spontaneous regression of cancer. Natl Cancer Inst Monogr, 1976. 44: p. 15-8.
30. Hunt, M. J., et al., Regression in basal cell carcinoma: an immunohistochemical analysis. Br J Dermatol, 1994. 130(1): p. 1-8.
31. Ferradini, L., et al., Analysis of T cell receptor variability in tumor-infiltrating lymphocytes from a human regressive melanoma. Evidence for in situ T cell clonal expansion. J Clin Invest, 1993. 91(3): p. 1183-90.
32. Ishizu, H., et al., Immune-mediated regression of ‘metastatic’ neuroblastoma in the liver. J Pediatr Surg, 1994. 29(2): p. 155-9; discussion 159-60.
33. Abubakr, Y. A., T. H. Chou, and B. G. Redman, Spontaneous remission of renal cell carcinoma: a case report and immunological correlates. J Urol, 1994. 152(1): p. 156-7.
34. Mentzer, S. J., Immunoreactivity in lung cancer. Chest Surg Clin N Am, 1995. 5(1): p. 57-71.
35. Zorn, E. and T. Hercend, A MAGE-6-encoded peptide is recognized by expanded lymphocytes infiltrating a spontaneously regressing human primary melanoma lesion. Eur J Immunol, 1999. 29(2): p. 602-7.
36. Inoue, T., et al., Spontaneous regression of merkel cell carcinoma: a comparative study of TUNEL index and tumor-infiltrating lymphocytes between spontaneous regression and non-regression group. J Dermatol Sci, 2000. 24(3): p. 203-11.
37. Nakai, T., T. Shimomura, and F. Hirokawa, Spontaneous regression of recurrent hepatocellular carcinoma after TAE: possible mechanisms of immune mediation. Int J Clin Oncol, 2001. 6(3): p. 149-52.
38. Kawai, K., et al., Enhancement of T cell proliferative response against autologous cancer cells of a metasatic renal cell carcinoma patient after unexplained regression. Int J Urol, 2004. 11(12): p. 1130-2.
39. Saleh, F., et al., Direct evidence on the immune-mediated spontaneous regression of human cancer: an incentive for pharmaceutical companies to develop a novel anti-cancer vaccine. Curr Pharm Des, 2005. 11(27): p. 3531-43.
40. Hamilton, D. H. and P. A. Bretscher, Different immune correlates associated with tumor progression and regression: implications for prevention and treatment of cancer. Cancer Immunol Immunother, 2008. 57(8): p. 1125-36.
41. Bowles, A. P., Jr. and E. Perkins, Long-term remission of malignant brain tumors after intracranial infection: a report of four cases. Neurosurgery, 1999. 44(3): p. 636-42; discussion 642-3.
42. Nakamura, Y., et al., Spontaneous remission of a non-small cell lung cancer possibly caused by anti-NY-ESO-1 immunity. Lung Cancer, 2009. 65(1): p. 119-22.
43. Lazarus, D. S., J. T. Kurnick, and R. L. Kradin, Alterations in pulmonary function in cancer patients receiving adoptive immunotherapy with tumor-infiltrating lymphocytes and interleukin-2. Am Rev Respir Dis, 1990. 141(1): p. 193-8.
44. Fisher, B., et al., Tumor localization of adoptively transferred indium-111 labeled tumor infiltrating lymphocytes in patients with metastatic melanoma. J Clin Oncol, 1989. 7(2): p. 250-61.
45. Hayakawa, K., et al., Study of tumor-infiltrating lymphocytes for adoptive therapy of renal cell carcinoma (RCC) and metastatic melanoma: sequential proliferation of cytotoxic natural killer and noncytotoxic T cells in RCC. J Immunother (1991), 1991. 10(5): p. 313-25.
46. Whiteside, T. L., Cancer therapy with tumor-infiltrating lymphocytes: evaluation of potential and limitations. In Vivo, 1991. 5(6): p. 553-9.
47. Rosenberg, S. A., The development of new cancer therapies based on the molecular identification of cancer regression antigens. Cancer J Sci Am, 1995. 1(2): p. 90-100.
48. Skornick, Y., S. Topalian, and S. A. Rosenberg, Comparative studies of the long-term growth of lymphocytes from tumor infiltrates, tumor-draining lymph nodes, and peripheral blood by repeated in vitro stimulation with autologous tumor. J Biol Response Mod, 1990. 9(4): p. 431-8.
49. Goedegebuure, P. S., et al., Adoptive immunotherapy with tumor-infiltrating lymphocytes and interleukin-2 in patients with metastatic malignant melanoma and renal cell carcinoma: a pilot study. J Clin Oncol, 1995. 13(8): p. 1939-49.

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

1. A fibroblast cell, said cell expressing a chimeric antigen receptor (CAR).

2. The cell of claim 1, wherein the chimeric antigen receptor comprises at least one antigen-specific targeting region, at least one transmembrane domain, and at least one intracellular signaling domain.

3. The cell of claim 2, wherein the antigen-specific targeting region is an scFv.

4. The cell of claim 2, wherein the antigen-specific targeting region targets CD19, TEM-1, TEM-2, TEM-3, TEM-4, TEM-5, TEM-6, TEM-7, TEM-8, ROBO-4, VEGRF2, CD109, survivin and/or CD93 antigen.

5. The cell of claim 2, wherein the intracellular signaling domain comprises part or all of an intracellular domain of TLR-4.

6. The cell of claim 2, wherein the intracellular signaling domain comprises a shRNA that encodes a transcript that generates at least one siRNA capable of inhibiting expression of HLA I and/or HLA II.

7. The cell of claim 1, wherein the cells comprise activity to elicit an immune reaction in an individual.

8. The cell of claim 7, wherein the activity comprises the ability to produce one or more cytokines at a level higher than that of fibroblasts that do not express the CAR.

9. The cell of claim 8, wherein the cytokine is selected from the group consisting of IL-12, IL-7, IL-15, IL-21, and a combination thereof.

10. The cell of claim 1, wherein the activity comprises the ability to stimulate a T cell response and/or an NK cell response.

11. The cell of claim 10, wherein the NK cells are CD94⁺ and/or CD117⁺ and/or CD161⁻ and/or NKG2D⁺ and/or NKp46⁺ and/or CD226⁺ and/or CD57⁺.

12. The cell of claim 1, wherein the cell has been exposed to one or more anti-apoptotic proteins.

13. The cell of claim 12, wherein the anti-apoptotic protein is STC-1, BCL-2, XIAP, Survivin, Bcl-2XL, GATA-4, FGF-2, HO-1, or a combination thereof.

14. The cell of claim 1, wherein the cell expresses one or more anti-apoptotic proteins.

15. The cell of claim 14, wherein the anti-apoptotic protein is STC-1, BCL-2, XIAP, Survivin, Bcl-2XL, GATA-4, FGF-2, HO-1, or a combination thereof.

16. The cell of claim 1, wherein the cell is from the skin, heart, blood vessels, bone marrow, skeletal muscle, liver, pancreas, brain, adipose tissue, foreskin, placental, and/or umbilical cord.

17. The cell of claim 1, wherein the cell is placental, fetal, neonatal, adult, or a mixture thereof.

18. The cell of claim 1, wherein the cell is human.

19. An isolated plurality of cells comprising a plurality of the cell from claim 1.

20. A method of treating a medical disease or condition in an individual, comprising the step of administering to the individual a therapeutically effective amount of a plurality of the fibroblast cells of claim 1.

21. The method of claim 20, wherein the medical disease or condition is cancer.

22. The method of claim 20, wherein the individual is given an additional cancer therapy.

23. The method of claim 22, wherein the additional cancer therapy is surgery, radiation, chemotherapy, hormone therapy, immune therapy, or a combination thereof.

24. The method of claim 20, further comprising the step of preparing the cell.

25. A method of stimulating T cells and/or NK cells, comprising the step of administering an effective amount of the cells of claim 1 to the T cells and/or NK cells.

26. The method of claim 25, wherein the method occurs ex vivo.

27. The method of claim 25, wherein the method occurs in vivo.

28. A method of enhancing activity of CAR-expressing cells in an individual, comprising the step of administering to the individual an effective amount of fibroblasts before, during, and/or after administering an effective amount of CAR-expressing cells.

29. The method of claim 28, wherein the CAR-expressing cells are immune cells, stem cells, fibroblasts, or a combination thereof.

30. The method of claim 28, wherein the CAR-expressing cells are modified to express one or more cytokines.

31. The method of claim 20, wherein the individual is provided an effective amount of one or more immune stimulators.

32. The method of claim 31, wherein the one or more immune stimulators is a toll like receptor agonist.

33. The method of claim 20, wherein the fibroblasts are autologous, allogeneic, and/or xenogenic with respect to the individual.
